(19) United States
(12) Patent Application Publication
(10) Pub. No.: US 2026/0159606 A1
(43) Pub. Date: Jun. 11, 2026

(54) Antivirals Against Human Parainfluenza Virus

(71) Applicants: VIB VZW, Gent (BE); Universiteit Gent, Gent (BE)

(72) Inventors: Xavier Saelens, Gent (BE); Iebe Rossey, Gent (BE); Bert Schepens, Gent (BE); Hans Nauwynck, Merelbeke (BE)

(21) Appl. No.: 19/114,917

(22) PCT Filed: Sep. 27, 2023
(86) PCT No.: PCT/EP2023/076723

§ 371 Date: Mar. 25, 2025

(30) Foreign Application Priority Data
Sep. 27, 2022 (EP) 22197948.7

(51) Int. Cl. C07K 16/40; A61K 39/00; A61P 31/14

(52) U.S. Cl. CPC C07K 16/40; A61P 31/14; A61K 2039/505; C07K 2317/24; C07K 2317/33; C07K 2317/565; C07K 2317/567; C07K 2317/569; C07K 2317/76; C07K 2317/92

(57) ABSTRACT

The invention relates to antigen-binding protein agents specifically binding the hemagglutinin-neuraminidase (HN) protein of human parainfluenza virus 3 (hPIV-3), with cross-reactivity for Sendai Virus (SeV) as well as human parainfluenza virus serotype 1 (hPIV-1) HN, resulting in a cross-neutralizing binding protein to act as a protection agent against parainfluenza infection and/or disease caused by such an infection. More specifically, the invention relates to a family of immunoglobulin single variable domains (ISVDs) capable of broadly blocking HN neuraminidase activity, thereby providing for a novel hPIV-1/-3 cross-neutralizing antiviral protein binding agent. The invention further relates to the use of said antigen-binding proteins in prevention or treatment of acute respiratory tract infections, croup, pneumonia, and exacerbations of chronic obstructive pulmonary disease.

### CROSS-REFERENCE TO RELATED APPLICATIONS

This application is a national phase entry under 35 U.S.C. § 371 of International Patent Application PCT/EP2023/076723, filed Sep. 27, 2023, designating the United States of America and published in English as International Patent Publication WO2024/068744 on Apr. 4, 2024, which claims the benefit under Article 8 of the Patent Cooperation Treaty to European Patent Application Serial No. 22197948.7, filed Sep. 27, 2022, the entireties of which are hereby incorporated by reference.

### INCORPORATION BY REFERENCE

The ST.26 XML Sequence listing named “10904US20260225substitutesequencelisting”, created on Feb. 26, 2026, and having a size of 79,850 bytes, is hereby incorporated herein by this reference in its entirety.

### FIELD OF INVENTION

The invention relates to antigen-binding protein agents specifically binding the hemagglutinin-neuraminidase (HN) protein of human parainfluenza virus 3 (hPIV-3), with cross-reactivity for Sendai Virus (SeV) as well as human parainfluenza virus serotype 1(hPIV-1) HN, resulting in a cross-neutralizing binding protein to act as a protection agent against parainfluenza infecton and/or disease caused by such an infection. More specifically, the invention relates to a family of immunoglobulin single variable domains (ISVDs) capable of broadly blocking HN neuraminidase activity, thereby providing for a novel hPIV-1/-3 cross-neutralizing antiviral protein binding agent. The invention further relates to the use of said antigen-binding proteins in prevention or treatment of acute respiratory tract infections, croup, pneumonia, and exacerbations of chronic obstructive pulmonary disease.

### INTRODUCTION

Human Parainfluenza viruses (hPIVs) are enveloped, single-stranded RNA viruses of the Paramyxoviridae family. They are the second most important cause of acute lower respiratory tract illness (LRTI) in the (very) young, only preceded by respiratory syncytial virus (1, 2, 3, 4). Specifically, in this age group, they are responsible for approximately 725.000 yearly hospitalizations due to LRTI and 34.400 yearly deaths worldwide (4). hPIV infection is also common in the elderly and in immune-compromised patients, often culminating in severe pneumonia and even mortality (6, 7, 22). Furthermore, hPIVs cause up to 75% of croup cases, can induce chronic obstructive pulmonary disease (COPD) exacerbations, and account for 2 to 11.5% of adult hospitalizations due to RTIs (5). hPIVs thus cause a significant disease burden. hPIVs are generally classified into four serotypes, denoted as hPIV-1 to -4. hPIV-1 and -3 are classified in the Respirovirus genus (and are also called Respirovirus 1 and 3), whereas hPIV-2 and -4 serotypes are classified as Rubulaviruses. Infection rates are serotype-specific and demonstrate distinct seasonal peaks depending on the region (8). In the United States, the majority of clinical cases is due to hPIV-3 (52%) and hPIV-1 (26%) (9). hPIV virions use two envelope proteins to infect host cells: the fusion protein (F) and the hemagglutinin-neuraminidase protein (HN). The HN protein binds sialic acids on the host epithelial cell surface, after which it triggers the associated viral F protein to insert it's fusion peptide in the host cell membrane.

This is associated with a conformational transition of the F protein from a globular prefusion state, via an extended intermediate conformation, to a postfusion state, ultimately resulting in fusion of the viral and host cell membrane and the creation of a fusion pore through which the viral genome can migrate into the cytoplasm of the infected cell (10). At current, there are no vaccines or antivirals available in the clinic to prevent or specifically treat disease caused by hPIV. Treatment is restricted to supportive and symptomatic care such as administration of oxygen, fever control, and in the case of mild to moderate croup, corticosteroids. The antiviral ribavirin, and/or intravenous immunoglobulins are sometimes used in immune compromised patients, but with poor support level (20). There is thus an urgent need for hPIV-directed antivirals to help control disease caused by these globally prevalent respiratory viruses.

Antibodies against viral F or HN proteins can be neutralizing and were found to protect against infection in rodents (24-26). Thus, antibodies against these envelope proteins may be valuable antiviral agents in reducing hPIV-associated disease burden. Several antibodies have already been described that specifically bind to the prefusion state of the hPIV-3 F protein (PIA174, PI3-A3, PI3-E12) (11, 12). HN-specific antibodies have also been isolated but serve mainly diagnostic or research purposes. These antibodies bind to hPIV-3 HN, to the HN protein of Sendai Virus (SeV), which is the murine variant of hPIV-1, or to both hPIV-3 and SeV HN (13, 14, 15, 19). Only few of these cross-binding antibodies were shown to neutralize one of both viruses, but all lack cross-neutralization activity (16, 19). The low conservation level between different hPIV serotype HN proteins provides for an additional barrier to provide new therapies which are broadly effective against more than one of the hPIV serotypes, while several serotypes cause disease.

### SUMMARY OF THE INVENTION

The invention relates to protein binding agents specifically interacting with the haemagglutinin-neuraminidase (HN) protein of human parainfluenza virus (hPIV) serotype 3 (hPIV-3). The binding agents disclosed herein are the result from initial selection of immunoglobulin single variable domain (ISVD) binders specific for hPIV-3 virions, for which their binding site was shown to be located on the HN protein, and, moreover, which comprise an antigen-binding domain which competes with Zanamivir for contacting the hPIV-3 HN. Unexpectedly, even though the amino acid sequence identity between hPIV serotype 1 and 3 HN is only 48%, cross-reactivity with Sendai as well as hPIV-1 HN was detected for most of the binders. Even more striking, the Sendai and/or hPIV-1 binding of the ISVDs resulted in potent neutralization of Sendai and hPIV-1 in a cell-based assay, and protection against Sendai virus in mice. The fact that the amino acid sequences of HN of hPIV serotype-1/-3 have a low sequence identity, which is even lower compared with HN of hPIV-2 and -4, which are descendent from different viral lineages, increases the bar to come up with new therapies that are broadly effective against more than one of the hPIV serotypes, while several serotypes cause disease. So, the cross-neutralizing agents described in the present invention provide for a first step towards building novel broadly effective respiratory antivirals.

In a first aspect, the invention relates to hPIV-3 HN-specific binders which are inhibitors of infection with both hPIV-3 and Sendai virus, the murine equivalent of hPIV-1. In a specific embodiment, the antigen-specific binder is of protein nature, herein called an antigen-binding protein, neutralizes at least hPIV-3 and Sendai virus, and specifically binds to or interacts with the hPIV-3 HN via its antigen-binding domain.

In a further embodiment said hPIV-3 HN-specific binding protein agent partly inhibits binding of Zanamivir to hPIV-3 HN, or preferably blocks or prohibits binding of Zanamivir to hPIV-3 HN, or most preferably outcompetes Zanamivir for binding to hPIV-3 HN. In a further embodiment, said antigen-specific binding protein neutralizes hPIV-3 and Sendai virus with a specific antagonist drug potency, referred to as half maximal inhibitory concentration, i.e. IC₅₀ value, of 1 μM or lower. In a specific embodiment, the half maximal inhibitory concentration of virus neutralization is 30 nM or lower.

A further embodiment relates to any of the hPIV-3 HN-specific protein binding agents as described herein and further specifically binding hPIV serotype 1 (hPIV-1) HN, even more preferably also capable of neutralizing hPIV-1 viral infection. In a specific embodiment, the hPIV-3 HN-binding agent as described herein, which neutralizes hPIV-3, Sendai virus and hPIV-1 has a potency corresponding to an IC₅₀ value of 1.5 μM or lower. In a specific embodiment, the IC₅₀ value of virus neutralization for all three viruses is 30 nM or lower, wherein said protein binding agent may be present in a mono- or multivalent format.

A further embodiment relates to any of the hPIV-3 HN-specific binding agent as described herein, which comprises an ISVD which interacts with the hPIV-3 HN via its single variable antigen-binding domain. In a specific embodiment, said hPIV-3 HN-specific binder comprises an ISVD amino acid sequence comprising CDRs as presented in any of SEQ ID NOs: 1-4, 70 or 71, wherein the CDRs are annotated according to Kabat, MacCallum, IMGT, AbM, or Chothia, as referred to herein, or as exemplified in FIG. 12. In another specific embodiment, said hPIV-3 HN-specific binding agent comprises an ISVD wherein
(a) CDR1 comprises SEQ ID NO: 17, CDR2 comprises SEQ ID NO: 21, and CDR3 comprises SEQ ID NO: 25; or
(b) CDR1 comprises SEQ ID NO: 18, CDR2 comprises SEQ ID NO: 22, and CDR3 comprises SEQ ID NO: 26; or
(c) CDR1 comprises SEQ ID NO: 19, CDR2 comprises SEQ ID NO: 23, and CDR3 comprises SEQ ID NO: 27; or
(d) CDR1 comprises SEQ ID NO: 20, CDR2 comprises SEQ ID NO: 24, and CDR3 comprises SEQ ID NO: 28.

In a further specific embodiment, the hPIV-3 HN-specific binding agent comprising an ISVD comprising any of the CDR1, CDR2, and CDR3 amino acid sequences as present in SEQ ID NOs: 1-4, with the CDRs being annotated according to Kabat, MacCallum, IMGT, AbM, or Chothia, or a CDR1 selected from SEQ ID NO:17-20, a CDR2 selected from SEQ ID NO:21-24, and a CDR3 selected from SEQ ID NO:25-28, further comprises the Framework regions (FRs) as presented in SEQ ID NO:42 for FR1, SEQ ID NO:43 for FR2, SEQ ID NO:44 for FR3, and SEQ ID NO: 45 for FR4. In another specific embodiment, the hPIV-3 HN-binding agents described herein comprises at least one ISVD comprising an amino acid sequence wherein the CDRs are annotated and provided as described herein above, and the FRs comprise the respective FR regions of SEQ ID Nos: 1-4, 70 or 71, specifically as selected for FR1 a sequence comprising any one of SEQ ID NOs:29-32, for FR2 a sequence comprising any one of SEQ ID NOs:33-36, for FR3 a sequence comprising any one of SEQ ID NOs:37-40, and FR4 comprising SEQ ID NO:41.

A further embodiment relates to the antigen-binding protein as described herein, specifically binding hPIV-3 HN, which comprises an ISVD comprising a sequence selected from the group of sequences of SEQ ID NOs: 1-4, or a functional variant of any one thereof with at least 90% amino acid sequence identity over the full length of the ISVD sequence wherein the non-identical amino acids are located in one or more FR residues. In a specific embodiment, the antigen-binding protein specific for hPIV-3 HN as described herein comprises an ISVD comprising a humanized variant of a sequence selected from the group of sequences of SEQ ID NOs: 1-4 or of any functional variant thereof as described herein. In a further specific embodiment, the antigen-binding protein as described herein comprises an ISVD which is a humanized variant of any one of SEQ ID NOs: 1-4, wherein at least one or more of the following amino acid residues (numbered according to Kabat) were substituted as follows: Q1D/E, Q5V, D21S, G33A, M71R, K76N, A79Y, K83R, and/or Q108L in the VHH provided in SEQ ID NO:1; Q1D/E, Q5V, G33A, D79Y, Q83R, and/or Q108L in the VHH provided in SEQ ID NO:2; and/or Q1D/E, Q5V, P40A, V60A, K83R, and/or Q108L in the VHH provided in SEQ ID NO:3; or specifically wherein the ISVD is a humanized variant comprising any one of SEQ ID NOs: 64-71.

In another embodiment, the hPIV-3 HN-binding protein as described herein is a multivalent or multispecific binding agent. The binding moieties within said multivalent or multispecific agent may be directly linked, or fused by a linker or spacer. In a specific embodiment, the multivalent or multispecific agent as described herein is an Fc fusion or an antibody. In a specific embodiment, the hPIV-3 HN-binding multivalent or multispecific agent is a bivalent or bispecific binder. In a further specific embodiment, the multivalent or multispecific agent that specifically interacts with the hPIV-3 HN protein comprises at least one of the sequences selected from the group of sequences of SEQ ID NOs: 1-4, or a functional variant of any one thereof with at least 90% sequence identity over the full length of the ISVD sequence wherein the non-identical amino acids are located in one or more FR residues, or a humanized variant of any one thereof. In a further specific embodiment, said multivalent or multispecific agent comprises a bivalent hPIV-3 HN-specific ISVD, such as for instance any one selected from the group of SEQ ID NOs: 5-8, or a homologues functional variant of any one thereof with at least 90% sequence identity over the full length of the sequence wherein the non-identical amino acids are located in one or more Framework residues, or a humanized variant of any one thereof, such as for instance any one of SEQ ID NOs: 52-54, 58 or 59.

In a further specific embodiment, said multivalent or multispecific antigen-binding agent comprises a humanized variant of any one of the SEQ ID NOs: 1-4, 70, 71, or comprises the humanized variants of any one of SEQ ID NOs: 64-71, such as the antigen-binding protein comprising any one of SEQ ID NOs: 52-61. In a further specific embodiment said multivalent or multispecific binding agent described herein comprises an Fc fusion of any one of the hPIV-3 HN-specific ISVDs as described herein, or of a humanized variant thereof, such as for instance comprising a sequence selected from any one of SEQ ID NOs:55-57 or 60-61.

Another embodiment relates to any of the hPIV-3 HN-specific mono- or multivalent, or mono- or multispecific binding agents as described herein, which are further labelled, tagged, or fused to a further moiety, such as another functional moiety. Said conjugated functional moiety may comprise a therapeutic moiety, or a half-life extension.

In a further aspect, the invention relates to an isolated nucleic acid molecule encoding the one or more binding agents described herein, or specifically encoding the multivalent or multispecific binding agents, as defined herein. Further aspects relate to a vector comprising said nucleic acid molecule, or hosts containing the vector, nucleic acid molecule or protein binding agent as described herein.

Another aspect relates to a composition, which may be a pharmaceutical composition, comprising at least one hPIV-3-specific binding agent as described herein, or at least one nucleic acid molecule or vector encoding said binding agent or agents as described herein, and said pharmaceutical composition optionally comprising a further therapeutically active agent, a diluent, an adjuvant, a carrier and/or an excipient. The invention likewise relates to any of the above-described (pharmaceutical) compositions, binding agents, nucleic acids, and/or recombinant vectors, for use as a medicament. The invention likewise relates to any of the above-described (pharmaceutical) compositions, binding agents, nucleic acids and/or recombinant vectors, for use in prophylactic or therapeutic treatment of a subject, wherein the subject preferably is a human. The invention likewise relates to any above-described (pharmaceutical) compositions, binding agents, nucleic acids and/or recombinant vectors, for use in the prevention or treatment of a human parainfluenza virus infection.

### DESCRIPTION OF THE FIGURES

FIGS. 1A and 1B. hPIV-3-neutralizing activity in serum samples of alpacas immunized with hPIV-3. Alpaca 1 (FIG. 1A) and alpaca 2 (FIG. 1B) were intranasally immunized with 6*10⁸ plaque-forming units (PFU) hPIV-3 on day 0, followed by two subcutaneous boosts with 2*10⁸ PFU hPIV-3 plus adjuvant on days 27 and 57. Serum was harvested pre-immunization and at different time-points after the first—immunization (2, 4, 6 and 8 weeks). Monolayers of Vero cells were then infected with hPIV-3-GFP in the presence of a threefold dilution series of the serum samples, starting from a tenfold dilution (as indicated on the X-axis). Pre-immune plasma from another alpaca was included as negative control (neg ctrl plasma). The GFP fluorescence signal in Vero cells was measured three days post-infection with a Tecan Infinite 200PRO M-Plex plate reader.

FIGS. 2A and 2B. Screening periplasmic extracts for VHHs with hPIV-3-neutralizing activity. A phage VHH-display library was generated from alpaca 2 and subjected to three panning rounds. Then, tenfold serial dilutions (starting from a tenfold dilution) of VHH-containing periplasmic extracts of individually picked E. coli clones were mixed with a constant amount of hPIV-3-GFP, and the mixtures were used to infect Vero cells at 37° C. After 3 days, the GFP fluorescence intensity of Vero cells was determined, and results are shown as the percentage of infection compared to hPIV-3-GFP-infected cells which did not receive periplasmic extracts. Alpaca hPIV-3 immune plasma was used as positive control (+), and a periplasmic extract from a clone producing an irrelevant VHH specific for the SARS-CoV2 receptor binding domain was used as negative control (−).

FIG. 3. Alignment of amino acid sequences of VHHs A2R3-55, A2R3-57, A2R3-59 and A2R3-60. Amino acid sequences are presented using Kabat numbering. The complementarity-determining regions (CDRs), annotated based on the Chothia region definition, are boxed.

FIG. 4. hPIV-3-binding VHHs bind to the hPIV-3 HN protein. HEK293T cells were transfected with expression vectors for hPIV-3 F, hPIV-3 HN or hPIV-1 HN, or with an empty control vector, in combination with a GFP-NLS expression vector to fluorescently track successfully transfected cells. One day later, cells were incubated with the indicated VHHs or control antibodies at 1 μg/ml, or with alpaca plasma samples diluted 50-fold, followed by incubation with respective primary detection antibodies (for VHH and plasma samples) and subsequent staining with respective secondary AF633-labeled detection antibodies. VHH/antibody binding to the cells was analyzed by flow cytometry based on AF633 fluorescence. Results are shown as the AF633 mean fluorescence intensity (MFI) ratio in GFP+ versus GFP− cells. hPIV3 plasma=alpaca hPIV-3 immune plasma as positive control; Ctrl plasma=plasma from an alpaca that had not been immunized with hPIV-3 as negative control; PIA174=positive control monoclonal control antibody specific for hPIV-3 F protein; Palivizumab=irrelevant monoclonal antibody as negative control.

FIG. 5. A2R3-57, A2R3-59 and A2R3-60 bind to hPIV-3 HN and hPIV-1 HN. HEK293T cells were transfected with expression vectors for hPIV-3 HN or hPIV-1 HN, or with an empty control vector, in combination with a GFP-NLS expression vector to fluorescently track successfully transfected cells. One day later, cells were incubated with the indicated VHHs at the indicated concentrations (in pg/ml), with F—VHH-CI184 as an irrelevant negative control VHH at 1 μg/ml, or with alpaca serum samples diluted fiftyfold, followed by incubation with respective primary detection antibodies (for VHH and serum samples) and subsequent staining with respective secondary AF633-labeled detection antibodies. VHH/antibody binding to the cells was analyzed by flow cytometry based on AF633 fluorescence. Results are shown as the AF633 mean fluorescence intensity (MFI) ratio in GFP+ versus GFP− cells. hPIV3 serum=hPIV-3 immune plasma as positive control; neg ctrl serum=plasma from another alpaca that has not been exposed to hPIV3, which served as a negative control.

FIG. 6. hPIV-binding VHHs bind to hPIV-3-, hPIV-1-, and Sendai Virus (SeV)-infected cells. Vero (E6) TMPRSS2 cells were infected with 1 multiplicity of infection (MOI) of hPIV-3-GFP, hPIV-1-GFP or SeV-GFP. One day later, cells were detached, stained with 1 μg/ml, 0.1 pg/ml, or 0.01 pg/ml of the indicated VHHs, and fixed with 1% PFA before further incubation with primary detection antibodies and subsequent staining with secondary AF633-labeled detection antibodies. VHH/antibody binding to the cells was analyzed by flow cytometry based on AF633 fluorescence. Results are shown as the AF633 mean fluorescence intensity (MFI) ratio in GFP+ versus GFP− cells.

FIGS. 7A and 7B. hPIV-3-, SeV- and hPIV-1-neutralizing activity of A2R3-55, A2R3-57, A2R3-59 and A2R3-60 monovalent VHHs. hPIV-3-GFP, SeV-GFP, or hPIV-1-GFP were preincubated with different concentrations of the indicated VHHs or monoclonal PIA174 control antibodies, and then used for infection of Vero cells (for hPIV-3 and SeV) or Vero (E6) TMPRSS2 cells (for hPIV-1). Two (hPIV-3 and hPIV-1) or three (SeV) days after infection, the GFP signal of infected cells was determined with a Tecan Infinite 200PRO M-Plex plate reader. The table shows the IC₅₀ values for hPIV-1, hPIV-3 and SeV as determined by the neutralization assay. PIA174=monoclonal control antibody specific for hPIV-3 F protein (11); F—VHH-CI184=irrelevant negative control VHH (21); No neut=no neutralization.

FIG. 8. VHHs can inhibit hPIV-1 and hPIV-3 neuraminidase activity. HEK293T cells were transfected with expression vectors for hPIV-3 HN or hPIV-1 HN. One day after transfection, cells were detached and incubated with VHHs (5 μg/mL), alpaca hPIV-3 immune serum (1/20 dilution) or with buffer diluted in McIlvain buffer at pH 5.4. MUNANA substrate was added to the cell-VHH suspensions and the fluorescent signal was determined every 2 minutes in a BMG FLUOstar OPTIMA reader during 1 hour at 37° C. Two buffer-only samples (i.e. transfected cells mixed with McIlvain buffer) were included to show the possible variability of the assay.

FIGS. 9A and 9B. Zanamivir competes with mono- and bivalent VHHs for binding to hPIV-3 HN. (FIG. 9A) HEK293T cells were transfected with expression vectors for hPIV-3 HN or hPIV-1 HN, or with an empty control vector, combined with a GFP-NLS expression vector. (FIG. 9B) HEK293T cells were transfected with an expression vector for hPIV-3 HN, or with an empty control vector, combined with a GFP-NLS expression vector. (FIGS. 9A-9B) One day after transfection, cells were incubated in 1% BSA with or without zanamivir (zana, 200 μg/ml in FIG. 9A), indicated concentrations in μg/ml in FIG. 9B). Next, the cells were stained with mono- or bivalent VHHs (0.1 μg/ml), alpaca hPIV-3 immune serum (hPIV3 serum, 1/50 dilution), or negative control alpaca plasma (Ctrl plasma, 1/50 dilution in FIG. 9A, 1/100 dilution in FIG. 9B), followed by incubation with respective primary detection antibodies and subsequent staining with respective secondary AF633-labeled detection antibodies. VHH/antibody binding to the cells was analyzed by flow cytometry based on AF633 fluorescence. Results are shown as the AF633 mean fluorescence intensity (MFI) ratio in GFP+ versus GFP− cells. F—VHH-CI184=irrelevant negative control VHH.

FIG. 10. hPIV-1-, hPIV-3-, and SeV-neutralizing activity of mono- and bivalent VHHs. hPIV-1-GFP, hPIV-3-GFP, and SeV-GFP were preincubated with different concentrations of the indicated VHHs (monovalent or bivalent), or monoclonal antibody PIA174, and then used for infection of Vero (E6) TMPRSS2 cells. Two days after infection, the GFP signal was measured with a Tecan Infinite 200PRO M-Plex plate reader. The table shows the IC₅₀ values in nM for hPIV-1, hPIV-3 and SeV as determined by the neutralization assay. F—VHH-CI184=irrelevant negative control VHH (21); PIA174=control monoclonal antibody specific for hPIV-3 F protein (11).

FIGS. 11A-11C. hPIV3 HN D275 and R277 residues are important for binding of A2R3-55, A2R3-57 and A2R3-59. FIG. 11A. Part (AA 271-280) of the hPIV-3 HN amino acid sequence aligned to hPIV-1 (AA 273-282) and SeV HN (AA 273-282). The conserved amino acids of interest are indicated in bold. FIG. 11B. Structure of hPIV-3 HN (PDB 1V3E) complexed with zanamivir (blue) and positions D275 and R277 indicated in orange (right). FIG. 11C. HEK293T cells were transfected with an expression vector for hPIV-3 HN (HN3), hPIV-1 HN (HN1), D275N- and R277T-mutant hPIV-3 HN (mut HN3), or N275D- and T277R-mutant hPIV-1 (mut HN1), or with an empty vector, combined with a GFP-NLS expression vector. One day after transfection, the cells were stained with VHHs (1 or 0.1 μg/ml), or with alpaca hPIV-3 immune or negative control (ctrl)plasma (1/100 or 1/1000 dilution), followed by incubation with respective primary detection antibodies and subsequent staining with respective secondary AF633-labeled detection antibodies. VHH/antibody binding to the cells was analyzed by flow cytometry based on the AF633 signal intensity. Results are shown as the AF-633 mean fluorescence intensity (MFI) ratio in GFP+ versus GFP− cells. F—VHH-CI184=irrelevant negative control VHH.

FIG. 12. Amino acid sequence of VHH-A2R3-60 with CDRs indicated according to the different annotations. Kabat numbering is used for numbering of the amino acid residues. The Complementary-determining-regions 1, 2 and 3 (CDR 1, 2, 3) are indicated as grey labelled boxed, according to AbM, MacCallum, Chothia, IMGT or Kabat annotation.

FIG. 13. Melting temperature (Tm) and aggregation temperature (Tagg) of recombinantly produced humanized bivalent (head-to-tail) VHHs and VHH-Fc fusions. Tagg was measured at OD266 nm and at OD473 nm. Samples were measured at a concentration of 1 mg/mL. Temperatures are provided in ° Celsius.

FIG. 14. Neutralization efficiency of bivalent VHHs for hPIV1, hPIV3 and Sendai Virus. hPIV-3-GFP, SeV-GFP, or hPIV-1-GFP were preincubated with different concentrations of the indicated recombinantly produced (h)VHHs, hVHH-Fcs or monoclonal PIA174 control antibody, and then used to infect Vero cells (for hPIV-3 and SeV) or Vero (E6) TMPRSS2 cells (for hPIV-1). Two days after infection, the GFP signal of infected cells was determined with a Tecan Infinite 200 PRO M-Plex plate reader. The table shows the ICsovalues for hPIV-1, hPIV-3 and SeV as determined by the neutralization assay. PIA174 =monoclonal negative control antibody specific for hPIV-3 F protein (11); F—VHH-CI184=irrelevant negative control VHH (21).

FIG. 15. Neutralization efficiency of G33A substituted VHH variants. hPIV-3-GFP, SeV-GFP, or hPIV-1-GFP were preincubated with different concentrations of the indicated (h)VHHs, hVHH-Fcs or monoclonal PIA174 control antibody, and then used for infection of Vero cells (for hPIV-3 and SeV) or Vero (E6) TMPRSS2 cells (for hPIV-1). Two days after infection, the GFP signal of infected cells was determined with a Tecan Infinite 200 PRO M-Plex plate reader. The table shows the ICsovalues for hPIV-1, hPIV-3 and SeV as determined by the neutralization assay. PIA174=monoclonal negative control antibody specific for hPIV-3 F protein (11); F—VHH-CI184=irrelevant negative control VHH (21).

FIGS. 16A and 16B. A2R3-59 and -60 cross bind to hPIV-4a and -4b. (FIG. 16A) HEK293T cells were transfected with expression vectors for hPIV-1/2/3/4a or 4b HN, in combination with a GFP-NLS expression vector to fluorescently track successfully transfected cells. One day later, cells were incubated with the indicated VHHs, hVHHs or hVHH-Fcs at 10 μg/ml or with F-VHH-CI184 as an irrelevant negative control VHH, or with alpaca serum samples diluted fiftyfold, followed by incubation with respective primary detection antibodies (for VHH, hVHH-Fc and serum samples) and subsequent staining with respective secondary AF633-labeled detection antibodies. VHH/hVHH-Fc/antibody binding to the cells was analyzed by flow cytometry based on AF633 fluorescence. Results are shown as the AF633 mean fluorescence intensity (MFI) ratio in GFP+ versus GFP− cells. (FIG. 16B) A dilution series of A2R3-h60-60 and h60-Fc was tested starting from 10 μg/ml to 0.001 μg/ml (lower graphs). Control sera (upper graph) used were guinea pig anti-hPIV-3/-2/-4a/-4b (BEI Resources) and goat anti-PIV-1 (ab20791, Abcam).

FIG. 17. A2R3-h60-Fc antibody protects DBA/2J mice against SeV infection. Groups of five DBA/2J mice were treated intraperitoneally with 100 μg of A2R3-hVHH-Fc purified antibody, immune serum or PBS one day before infection with 1000 PFU SeV/52. Mice were weighed daily. Five days after infection the pulmonary SeV load was determined by plaque assay (dashed line represents the detection limit) or by quantification of the amount of lung viral RNA using RT-qPCR. Horizontal lines indicate medians. * P<0.05, **P<0.01 (Mann-Whitney U-test).

FIG. 18. A2R3-60-60 and A2R3-h60-Fc bind to recombinant hPIV-1 and -3 HN. ELISA plates were coated with hPIV-1 HN linked to either dGCN4 or tetraGCN4, or hPIV-3 HN linked to dGCN4. The plates were incubated with a 1/3 dilution series of either A2R3-60-60 or A2R3-h60-Fc. The OD₄₅₀ values are depicted.

### DETAILED DESCRIPTION

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the 10488-10904-US same meaning as commonly understood by one of ordinary skill in the art (e.g. in molecular biology, biochemistry, structural biology, and/or computational biology).

‘Nucleotide sequence’, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. By “nucleic acid construct” or “construct sequence(s)” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” or a “nucleic acid molecule encoding” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences orgenomic DNA, while introns may be present as well under certain circumstances. An “expression vector” comprises an expression cassette which in turn comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation, and also myristoylation. Based on the amino acid sequence and the modifications, the atomic or molecular mass or weight of a polypeptide is expressed in (kilo)dalton (kDa). A “protein domain” is a distinct functional and/or structural unit in a protein. Usually a protein domain is responsible for a particular function or interaction, contributing to the overall role of a protein. Domains may exist in a variety of biological contexts, where similar domains can be found in proteins with different functions.

By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or VHH as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

The term “linked to”, or “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. Preferably, the percentage of identity is calculated over a window of the full-length sequence referred to. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity, which is hereby defined as a ‘functional variant’. A functional variant thus also refers to variants comprising one or more substitutions or mutations, resulting in a homologue, preferably of at least 70%, at least 80%, or at least 90% amino acid identity, wherein the functionality is retained or at least similar as compared to the wild type protein or reference protein.

The term “wild type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant”, “engineered” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. A functional variant thus also refers to ‘variants’ as comparted to the wild type, though with the limitation that such a functional variant has retained function and properties relevant for its function. With ‘function’ in view of the present disclosure is referred for instance to the function of the VHH or VHH-based product, which preferably is the specificity for HN binding, as well as the neutralizing capacity of hPIV or Sendai virus.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding.

A “binding agent”, or “agent” as used interchangeably herein, relates to a molecule that is capable of binding to another molecule, via a binding region or binding domain located on the binding agent, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. A “protein binding agent” is a binding agent of protein nature.

The term “binding pocket” or “binding site” refers to a region of a molecule or molecular complex, that, as a result of its shape and charge, favourably associates with another chemical entity or binding domain, such as a compound, proteins, peptide, antibody or Nb, among others. For antibody-related molecules, the term “epitope” or “conformational epitope” is also used interchangeably herein. The human PIV HN protein herein described comprises a binding pocket or binding site which includes, but is not limited to a Nanobody binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

The term “epitope” refers to a region of a molecule or molecular complex, that, as a result of its shape and charge, favourably associates with another chemical entity or binding domain, such as a compound, proteins, peptide, antibody or Nb, among others. An “epitope”, as used herein, thus refers to an antigenic determinant of a polypeptide, constituting a binding site or binding pocket on a target molecule, such as hPIV-3 HN. Said epitope may comprise at least one amino acid that is essential for binding the binding agent, though preferably comprise at least 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance, cryo-EM, or other structural analyses.

The term “antibody” refers to an immunoglobulin (Ig) molecule or a molecule comprising an immunoglobulin (Ig) domain, which specifically binds with an antigen. “Antibodies” can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more complementarity determining regions (CDRs) accounting for such specificity, typically at least 3 CDRs, or in conventional antibodies, defined by 6 CDRs. Non-limiting examples of active antibody fragments include immunoglobulin domains, Fab, F(ab)′2, scFv, heavy-light chain dimers, immunoglobulin single variable domains (ISVDs), Nanobodies (or VHH antibodies), domain antibodies, and single chain structures, such as a complete light chain or complete heavy chain. The term “antibody fragment” and “active antibody fragment” or “functional variant” as used herein refers to a protein comprising an immunoglobulin domain or an antigen-binding domain capable of specifically binding hPIV HN. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, binds to the respective epitope of an antigen by a pair of (associated) immunoglobulin domains such as light and heavy chain variable domains, i.e., by a VH-VL pair of immunoglobulin domains, which jointly bind to an epitope of the respective antigen. An “immunoglobulin single variable domain (ISVD)” as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. For numbering of the amino acid residues of an IVD different numbering schemes can be applied. For example, numbering can be performed according to the AHo numbering scheme for all heavy (VH) and light chain variable domains (VL) given by Honegger, A. and Pluckthun, A. (J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231(1-2), J Immunol Methods. 1999. It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. Determination of CDR regions may also be done according to different methods, such as the designation based on contact analysis and binding site topography as described in MacCallum et al. (J. Mol. Biol. (1996) 262, 732-745). Or alternatively the annotation of CDRs may be done according to AbM (AbM is Oxford Molecular Ltd.'s antibody modelling package as described on http://www.bioinf.org.uk/abs/index.html), Chothia (Chothia and Lesk, 1987; Mol Biol. 196:901-17), Kabat (Kabat et al., 1991; 5th edition, NIH publication 91-3242), IMGT (LeFranc, 2014; Frontiers in Immunology. 5 (22): 1-22)). Those annotations exist for numbering amino acids in immunoglobulin protein sequences, though in the present application solely the Kabat numbering is used, or the specific SEQ ID numbering, as indicated. Said annotations further include delineation of CDRs and framework regions (FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target.

Immunoglobulin single variable domains (ISVDs) such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution. Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions, such as at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat). Humanization typically only concerns substitutions in the FR and not in the CDRs, as this could/would impact binding affinity to the target and/or potency.

‘Antigen-binding proteins’ or ‘antigen-binding domains’ as described herein may be derived from an antibody as described herein, or may be derived from alternative antigen-binding proteins with a different fold, so non-immunoglobulin binding proteins such as but not restricted to avimers, DARPins, alphabodies, affitins, nanofitins, anticalins, monobodies and lipocalins.

The term ‘antibody’ or ‘Fc-fusion’ as used herein further refers to the genetic linking or fusion of antigen-binding fragments or antigen-binding domains with an Fc constant domain as to obtain dimers forming an antibody structure when expressed in a recombinant host. In particular, antibody fragments, or single domain antibodies such as ISVDs may be C-terminally fused to the N-terminus of an Fc domain, preferably via a linker or hinge region. Alternatively, antibody fragments, or single domain antibodies such as ISVDs, may be fused at the N-terminus to the C-terminal end of an Fc domain, preferably via a linker or hinge region. Said single domain antibody or ISVD fused to said Fc may comprise one or more VHHs or Nbs, as described herein.

“Fc domains” or “Fc-regions” or “Fc-tails”, as interchangeably used herein, and refer to the single Fc chain and/or the dimeric Fc domain of an Fc-containing proteins. Specifically in antibodies, said Fc domain is thus responsible for antibody function, and Antibody Fc engineering stands for engineering functions of antibodies, which are effector functions, such as antibody-dependent cellular cytotoxicity (ADCC) and antibody-dependent cellular phagocytosis (ADCP), and controlling serum half-life. Engineered Fc domains may therefore be present in the form of mutants or variants containing amino acid substitutions, insertions or deletions as to allow different modifications of the Fc in post-translational modifications, dimerization behavior, effector function, serum half life, among others. To indicate the variations present in Fc domains based on the sequence of naturally occurring IgGs, conventional antibody numbering annotations are known in the art, such as for instance IMGT numbering (LeFranc, 2014; Frontiers in Immunology. 5 (22): 1-22), Kabat numbering (Kabat, E. A. et al., Sequences of proteins of immunological interest. 5th Edition—US Department of Health and Human Services, NIH publication n° 91-3242, pp 662,680,689 (1991)), or preferably used herein EU numbering (Edelman et al. (1969). The covalent structure of an entire gammaG immunoglobulin molecule. Proc Natl Acad Sci USA.; 63:78-85).

As used herein, a “therapeutically active agent” or “therapeutically active composition” means any molecule or composition of molecules that has or may have a therapeutic effect (i.e. curative or prophylactic effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition (described below), of the invention may act as a therapeutically active agent, when beneficial in treating patients infected with or at risk of infection with hPIV. The therapeutically active agent/binding agent or therapeutically active composition may include an agent comprising an ISVD specifically binding the hPIV HN and/or may contain or be coupled to additional “functional groups”, interchangeably called “functional moieties” herein, which are advantageous when administered to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the hPIV HN and one against a serum protein such as albumin aiding in prolonging half-life) or into fusions of antibody fragments, in particular immunoglobulin single variable domains, with peptides (for example, a peptide against a serum protein such as albumin).

As used herein, the terms “determining,” “measuring,” “assessing,”, “identifying”, “screening”, “addressing”, “testing”, and “assaying” are used interchangeably and include both quantitative and qualitative determinations. “Similar” as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, or is expected to be at high risk of developing a disease or disorder, in particular a disease or disorder as disclosed herein, also designated “patient” herein. However, it will be understood that the aforementioned terms do not imply that symptoms are present.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring, herein referred to as “prevention”.

A “composition” relates to a combination of one or more active molecules, and may further include buffered solutions and/or solutes such as pH buffering substances, water, saline, physiological salt solutions, glycerol, preservatives, etc. for which a person skilled in the art is aware of the suitability to obtain optimal performance. Suitable conditions as used herein could also refer to suitable binding conditions, for instance when Nbs or test compounds are aimed to bind hPIV HN.

A “pharmaceutical composition” is a therapeutically active composition comprising the one or more antigen-binding agents or therapeutically active agents or therapeutically active compositions and optionally comprising a carrier, diluent or excipient. A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent” includes vehicles such as water, saline, physiological salt solutions, glycerol, ethanol, etc. Auxiliary substances such as wetting or emulsifying agents, pH buffering substances, or preservatives may be included in such vehicles. A pharmaceutically effective amount of polypeptides, or conjugates of the invention and a pharmaceutically acceptable carrier is preferably that amount which produces a result or exerts an influence on the particular condition being treated. For therapy, the pharmaceutical composition of the invention can be administered to any patient in accordance with standard techniques. The administration can be by any appropriate mode, including orally, parenterally, topically, nasally, ophthalmically, intrathecally, intracerebroventricularly, sublingually, rectally, vaginally, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN™, PLURONICS™ or PEG and the like.

### DETAILED DESCRIPTION

Paramyxovirus belongs to the Paramyxovirinae subfamily of the Paramyxoviridae family, and represent enveloped viruses with a single strand of negative sense RNA of approximately 13 to 19 kb as a genome. Examples of paramyxoviruses include, among others, human parainfluenza virus (hPIV) including distinct serotypes 1, 2, 3, and 4 (hPIV-1, hPIV-2, hPIV-3, hPIV-4, respectively, with hPIV-4 being subdivided in subtypes 4A and 4B), mouse parainfluenza type 1 (Sendai virus, SeV) which is the animal counterpart of hPIV-1 (Chancock et al., Parainfluenza Viruses, Knipe et al. (Eds.), pp. 1341-1379, Lippincott Williams & Wilkins, Philadelphia, 2001). hPIV-1, hPIV-3 and Sendai Virus are classified in the genus Respirovirus, whereas hPIV-2 and hPIV-4 are classified in the genus Rubulavirus.

In a first aspect of the invention, the binding agents provided herein specifically bind hPIV serotype-3 virions, more specifically via binding to an epitope on the HN protein, and are capable of neutralizing hPIV-3 as well as Sendai virus.

The term “human parainfluenza virus or hPIV serotype x” or “hPIV-x” as used to herein, thereby thus refers to an isolate, clone, recombinant, or variant of human parainfluenza virus serotype x of the Paramyxovirinae subfamily. A “naturally occurring” isolate or “wild type” hPIV-x is a virus isolated from a natural source or has the sequence of a hPIV-x isolated from a natural source. Naturally occurring isolates may differ from one another in sequence. “Recombinant hPIV-x” refers to virus derived from a polynucleotide that has been constructed to encode a hPIV genome or antigenome, and may include a sequence of a wild type or variant hPIV-x. In some embodiments, the recombinant hPIV-x comprises an expression vector.

As used herein, a “neutralizing” agent refers to an agent that binds a target protein on a virion, specifically as described herein the protein binding agent specifically binds hPIV-3 HN protein, wherein upon said binding the agent inhibits or reduces at least one biological activity of the virion (e.g. entry of the hPIV-3 virion into a host cell), thereby having the capacity to be “neutralizing” to the extent that such viral activity is affected, optimally thereby preventing or reducing viral infection of the host. With inhibiting or reducing the biological activity as to obtain a neutralizing effect, it is meant herein that the agent is inhibits, reduces or alters the activity by showing an extent of at least 10% lower activity as compared to a virion or target protein in the absence of the binding agent, or in the presence of a control agent, or preferably of at least 20% lower activity, or at least 30%, at least 40%, at least 50%, at least 60%, at least 70%, at least 80%, at least 90%, at least 95% lower activity, or undetectable levels of activity, as compared to a control or vehicle.

In one embodiment an antigen-binding protein is described, wherein antigen refers to the target protein, specifically the HN protein, which is bound at a specific binding site or epitope, by a proteinaceous binding agent, as described herein.

The term “competing binding agent” or “agent competing for” or “agent competing with” refers to an agent which specifically binds to an antigen (e.g. Zanamivir binding to hPIV-3 HN) whereby said binding resulting in an inhibition or blocking of another binding agent (e.g. a VHH) to that same antigen or target. The first and second agent may bind to the same epitope. Alternatively, the first and second agent may bind to different, but overlapping epitopes such that binding of one inhibits or blocks the binding of the other one, e.g. by steric hindrance. Moreover, the first and second binding agent may bind to a totally different epitope, but in an allosteric mode, thereby changing the conformation of the antigen, and thereby blocking further binding of the second agent. Competition may be measured by methods known in the art. With ‘competing’ is meant that the binding of one HN binder as described herein is reduced with at least 30%, or at least 50%, or preferably at least 80% in strength in the presence of said competing binding agent.

In a specific embodiment, the antigen-binding protein specifically binding hPIV-3 HN, capable of neutralizing hPIV-3 and Sendai virions, has a potency of at least 1 μM or lower as IC₅₀ concentration, wherein said IC₅₀ value thus represents the inhibitory effect of the binding agent on virus infectivity, and specifically defines the concentration of the antigen-binding protein required to neutralize 50% of the infection, thereby allowing comparison between different treatments. In another embodiment the antigen-binding proteins as described herein has an IC₅₀ value of 1 μM, or 500 nM, 400 nM, 300 nM, 200 nM, 100 nM, 80 nM, 60 nM, 50 nM, 40 nM, 30 nM, 20 nM, 10 nM, or is in the picomolar range for neutralizing hPIV-3 and Sendai virus. In a further specific embodiment, the antigen-binding proteins as described herein has an IC₅₀ value of 1 μM, or 500 nM, 400 nM, 300 nM, 200 nM, 100 nM, 80 nM, 60 nM, 50 nM, 40 nM, 30 nM, 20 nM, 10 nM, 1 nM, or is in the picomolar range for neutralizing hPIV-3 and Sendai virus, as determined in a neutralization assay as exemplified herein, using Vero cells or Vero (E6) TMPRSS2 cells. In a further embodiment, the antigen-binding protein as described herein comprises a bivalent hPIV-3 HN binder and has a potency of 50 nM, 40 nM, 30 nM, 20 nM, 10 nM, 1 nM, or picomolar IC₅₀ concentration in neutralizing Sendai and hPIV-3, as determined in a neutralization assay.

In another embodiment, said antigen-binding protein as described herein is specifically binding hPIV-3 HN, as well as Sendai virus HN, as well as hPIV-1 HN, and neutralizes each of those viruses hPIV-3, Sendai and hPIV-1. In a more specific embodiment, the antigen-binding protein specifically binding hPIV-3 HN, capable of neutralizing hPIV-3, hPIV-1 and Sendai virions, has a potency of at least 1.5 μM or lower as IC₅₀ concentration. In another embodiment the antigen-binding proteins as described herein has an IC₅₀ value of 1.5 μM, 1.3 μM, 1.2 μM, 1.1 μM, 1 μM, or 500 nM, 400 nM, 300 nM, 200 nM, 100 nM, 80 nM, 60 nM, 50 nM, 40 nM, 30 nM, 20 nM, 10 nM, or is in the picomolar range for neutralizing hPIV-3, hPIV-1 and Sendai virus. In a further specific embodiment, the antigen-binding proteins as described herein has an IC₅₀ value of 1.5 μM, 1.3 μM, 1.2 μM, 1.1 μM, 1 μM, or 500 nM, 400 nM, 300 nM, 200 nM, 100 nM, 80 nM, 60 nM, 50 nM, 40 nM, 30 nM, 20 nM, 10 nM, 1 nM, or is in the picomolar range for neutralizing hPIV-3, hPIV-1 and Sendai virus, as determined in a neutralization assay as exemplified herein, using Vero cells or Vero (E6) TMPRSS2 cells. In a further embodiment, the antigen-binding protein as described herein comprises a bivalent hPIV-3 HN binder and has a potency of 50 nM, 40 nM, 30 nM, 20 nM, 10 nM, 1 nM, or picomolar IC₅₀ concentration in neutralizing Sendai, hPIV-1 and hPIV-3, as determined in a neutralization assay.

In a further embodiment, any of the above antigen-binding proteins with the potency as referred to as IC₅₀ value as indicated, comprises an immunoglobulin single variable domain (ISVD) which comprises the antigen-binding domain specifically binding the HN protein, in particular the hPIV-3 and Sendai HN protein, more preferably as well as the hPIV-1 HN protein.

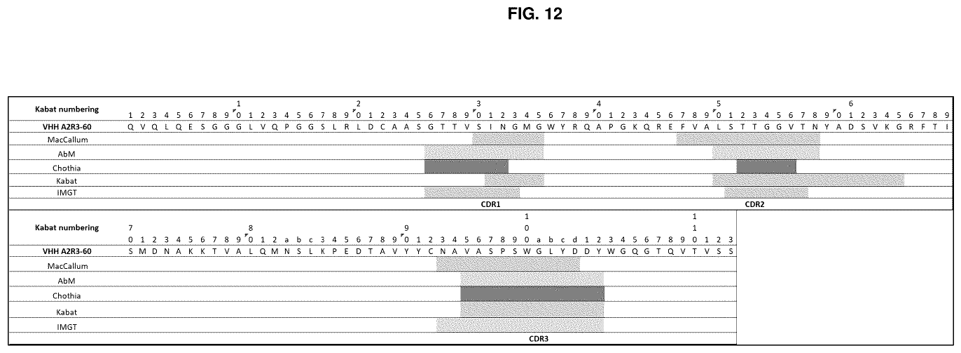

In a further aspect of the invention, the antigen-binding protein comprises an ISVD which specifically binds the hPIV-3 HN protein through binding of the CDRs as depicted in any one of SEQ ID NO:1-4, more specifically wherein said CDR1, CDR2, and CDR3 are defined by anyone of the annotations known in the art, and as provided for VHH A2R3-60 in FIG. 12 further herein, specifically according to MacCallum, Kabat, Chothia, IMGT or AbM annotation.

The CDR region annotation for each VHH sequence described herein according to Chothia (Chothia and Lesk, 1987; J Mol Biol. 196:901-17) is shown in the sequences listed herein (Table 1). Alternatively, slightly different CDR annotations known in the art may be applied here and relate to the AbM (AbM is Oxford Molecular Ltd.'s antibody modelling package as described on http://www.bioinf.org.uk/abs/index.html), IMGT (LeFranc, 2014; Frontiers in Immunology. 5 (22): 1-22), Kabat (Kabat et al., 1991; Sequences of Proteins of Immunological Interest. 5th edition, NIH publication 91-3242), or MacCallum et al. (J. Mol. Biol. (1996) 262, 732-745) annotation, which are all applicable to identify the CDR regions of the ISVDs as disclosed herein for SEQ ID NO: 1-4, 70 and 71. It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering, see for instance FIG. 12, where Kabat numbering is indicated for VHH A2R3-60). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

VHHs or Nbs are often classified in different sequences families or even superfamilies, as to cluster the clonally related sequences derived from the same progenitor during B cell maturation (Deschaght et al. 2017. Front Immunol. 10; 8:420). This classification is often based on the CDR sequence of the Nbs, and wherein for instance each Nb family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, having the same effect or functional impact.

In a specific embodiment the hPIV-3 HN-specific binding agent as described herein, comprises an ISVD which interacts with the hPIV-3 HN via its single variable antigen-binding domain, wherein said at least one ISVD amino acid sequence comprises CDRs as presented in any of SEQ ID NOs: 1-4, 70 or 71, representing the monovalent VHHs of the identified VHH family comprising A2R3-60, A2R3-59, A2R3-57, and A2R3-55, and A2R3-60(G33A), A2R3-59(G33A), resp., and wherein the CDRs are annotated according to Kabat, MacCallum, IMGT, AbM, or Chothia, as referred to herein, or as exemplified in FIG. 12. In another specific embodiment, said hPIV-3 HN-specific binding agent comprises an ISVD wherein CDR1 is defined by anyone of SEQ ID NOs: 17-20, CDR2 by anyone of SEQ ID NOs:21-24, and CDR3 by anyone of SEQ ID NOs: 25-28.

In a further specific embodiment, the hPIV-3 HN-specific binding agent comprising an ISVD comprising any of the CDR1, CDR2, and CDR3 amino acid sequences as present in SEQ ID NOs: 1-4, 70 or 71, with the CDRs being annotated according to Kabat, MacCallum, IMGT, AbM, or Chothia, or a CDR1 selected from SEQ ID NO:17-20, a CDR2 selected from SEQ ID NO:21-24, and a CDR3 selected from SEQ ID NO:25-28, further comprising Framework regions (FRs) as presented in:
SEQ ID NO:42 for FR1, comprising xVQLxESGGGLVQxGGSLRLxCAAS, wherein the position shown as x1 may be a Q, D, or E; the position shown as x5 may be Q or V; the position shown as x14 may be P or A, and the position shown as x21 may be D or S; and
SEQ ID NO:43 for FR2, comprising xMGWxRQxPGKQRExVAxx, wherein the position shown as x1 may be G or A; the position shown as x5 may be Y or S; the position shown as x8 may be A or P; the position shown as x15 may be F or L; the position shown as x18 may be L, A, or T; and the position shown as x19 may be S or I;
SEQ ID NO:44 for FR3, comprising TNYxxSVKGRFTISxDNAKxTVxLQMNSLxPEDTAVYYCxx, wherein the position shown as x4 may be A or V; the position shown as x5 may be D or V; the position shown as x15 may be M or R; the position shown as x20 may be K or N; the position shown as x23 may be A, D or Y; the position shown as x30 may be K, Q, or R; the position shown as x40 may be N, Y, or A; the position shown as x41 may be A, V, or L;
and SEQ ID NO: 45 for FR4, comprising WGQGTxVTVSS, wherein x is Q or L.

In another specific embodiment, the hPIV-3 HN-binding agents described herein comprises at least one ISVD comprising an amino acid sequence wherein the CDRs are annotated and provided as described herein above, and the FRs comprise the respective FR regions of SEQ ID Nos: 1-4, specifically as selected for FR1 a sequence comprising any one of SEQ ID NOs:29-32, for FR2 a sequence comprising any one of SEQ ID NOs:33-36, for FR3 a sequence comprising any one of SEQ ID NOs:37-40, and FR4 comprising SEQ ID NO:41.

A further embodiment relates to the antigen-binding protein as described herein, specifically binding hPIV-3 HN, which comprises an ISVD comprising a sequence selected from the group of sequences of SEQ ID NOs: 1-4, or a functional variant of any one thereof with at least 90%, or 95%, or 99% amino acid sequence identity over the full length of the ISVD sequence wherein the non-identical amino acids are located in one or more Framework residues. In a specific embodiment, the antigen-binding protein specific for hPIV-3 HN as described herein comprises an ISVD comprising a humanized variant of a sequence selected from the group of sequences of SEQ ID NOs: 1-4 or of any functional variant thereof as described herein.

Humanized and/or functional variants are obtained as described herein, and are based on primary sequence alignment with the human IGHv3 coding sequence, to substitute one or more key residues of the alpaca-derived framework regions of the VHHs, followed by biophysical analysis of the resulting VHHs after recombinant production. Specifically, the stability and neutralizing properties of the resulting VHHs are analysed. Moreover, said original and/or humanized variant sequence can be fused directly or via a linker, as to provide for (humanized) bivalent VHH variants, as tandem repeats, or head-to-tail fusion, as interchangeably used herein. Alternatively, they may be additionally fused to an Fc tail, more specifically a human IgG1 Fc.

So, in another embodiment the hPIV-3 HN-binding protein as described herein is a multivalent or multispecific binding agent. The binding moieties within said multivalent or multispecific agent may be directly linked, or fused by a linker or spacer. The composition or binding agent(s) as described herein may appear in a “multivalent” or “multispecific” form and thus be formed by bonding, chemically or by recombinant DNA techniques, together two or more identical or different binding agents. Said multivalent forms may be formed by connecting the building blocks directly or via a linker, or through fusing the building block(s) with an Fc domain encoding sequence. Non-limiting examples of multivalent constructs include “bivalent” constructs, “trivalent” constructs, “tetravalent” constructs, and so on. The immunoglobulin single variable domains comprised within a multivalent construct may be identical or different, preferably binding to the same or overlapping binding site. In another particular embodiment, the binding agent(s) of the invention are in a “multispecific” form and are formed by bonding together two or more building blocks or agents, of which at least one binds to hPIV HN, as shown herein, and at least one binds to a further target or alternative molecule, such as HN derived from a different hPIV serotype, so when present in multispecific fusion, presenting a binding agent or composition that is capable of specifically binding both epitopes or targets, thus comprising binders with a different specificity. Non-limiting examples of multi-specific constructs include “bispecific” constructs, “trispecific” constructs, “tetraspecific” constructs, and so on. To illustrate this further, any multivalent or multispecific (as defined herein) form of the invention may be suitably directed against one or more different epitopes on the same hPIV HN antigen, or on epitopes of hPIV HN proteins from different serotypes, in particular from serotype-1, -3, and/or -2, and/or -4, or may be directed against two or more different antigens, for example one building block against hPIV HN and one building block as a half-life extension against Serum Albumin or SpA, or another target. Multivalent or multi-specific ISVDs of the invention may also have (or be engineered and/or selected for) increased avidity and/or improved selectivity for the desired hPIV HN interaction, and/or for any other desired property or combination of desired properties that may be obtained by the use of such multivalent or multispecific immunoglobulin single variable domains. Upon binding hPIV HN, said multi-specific or multivalent binding agent may have an additive or synergistic impact on the binding and/or therapeutic effect on hPIV HN, such as an increase in its potency for blocking or inhibiting viral entry. In another embodiment, the invention provides a polypeptide comprising any of the immunoglobulin single variable domains according to the invention, either in a monovalent, multivalent or multispecific form. Thus, polypeptides comprising monovalent, multivalent or multispecific nanobodies are included here as non-limiting examples. The multivalent or multispecific binders or building blocks may be fused directly or fused by a suitable linker, as to allow that the at least two binding sites can be reached or bound simultaneously by the multivalent or multispecific agent.

Alternatively, at least one ISVD as described herein may be fused at its C-terminus to an Fc domain, for instance an Fc-tail of an Ig, resulting in an antigen-binding protein of bivalent format wherein two of said VHH-Ig Fcs, or humanized forms thereof, form a heavy chain only-antibody-type molecule through disulfide bridges in the hinge region of the Fc part, called “Fc fusion” herein. In a specific embodiment, the multivalent or multispecific agent as described herein is an Fc fusion or an antibody. Another embodiment comprises a humanized ISVD specifically binding hPIV-3 HN as described herein, comprised in a multivalent or multispecific agent, which may be provided as a humanized ISVD-IgG fusion, and which may further include but is not limited to the use of IgG humanization variants known in the art, such as C-terminal deletion of Lysine, alteration or truncation in the hinge region, LALA or LALAPG mutations as described, among other substitutions in the IgG sequence. In an alternative setup, the “Fc fusion” is designed by linking the C-terminus of such a bivalent or bispecific binder fused by a linker to an Fc domain, which then upon expression in a host forms a multivalent or multispecific-antibody-type molecule through disulfide bridges in the hinge region of the Fc part.

In a specific embodiment, the hPIV-3 HN-binding multivalent or multispecific agent is a bivalent or bispecific binder. In a further specific embodiment, the multivalent or multispecific agent that specifically interacts with the hPIV-3 HN protein comprises at least one sequence selected from the group of sequences of SEQ ID NOs: 1-4, 70, or 71, or a functional variant of any one thereof with at least 90%, or 95%, or 99% sequence identity over the full length of the ISVD sequence wherein the non-identical amino acids are located in one or more Framework residues, or a humanized variant of any one thereof.

Examples of the possible humanization variants of the monovalent VHHs have been disclosed in a non-limiting manner herein, wherein said humanized variant of the ISVD of said antigen-binding protein comprises: at least one of the following substitutions in SEQ ID NO:1: Q1D/E, Q5V, D21S, G33A, M71R, K76N, A79Y, K83R, Q108L; at least one of the following substitutions in SEQ ID NO:2: Q1D/E, Q5V, G33A, D79Y, Q83R, Q108L; at least one of the following substitutions in SEQ ID NO:3: Q1D/E, Q5V, P40A, V60A, K83R, Q108L; or a humanized variant comprising any one of SEQ ID NOs: 64-71.

In a further specific embodiment, said multivalent or multispecific agent comprises a bivalent hPIV-3 HN-specific ISVD, such as for instance any one selected from the group of SEQ ID NOs: 5-8, or a homologues functional variant of any one thereof with at least 90%, or 95%, or 99% sequence identity over the full length of the sequence wherein the non-identical amino acids are located in one or more framework residues, or a humanized variant of any one thereof, such as for instance any one of SEQ ID NOs: 52-54, 58 or 59, or a homologue with at least 90%, or 95%, or 99% sequence identity over the full length of the sequence wherein the non-identical amino acids are located in one or more framework residues, thereof.

In a further specific embodiment, said multivalent or multispecific antigen-binding agent comprises a humanized variant of any one of the SEQ ID NOs: 1-4, 70, 71, or comprises the humanized variants of any one of SEQ ID NOs: 64-71, such as for instance but not limited to the antigen-binding protein comprising any one of SEQ ID NOs: 52-61. In a further specific embodiment said multivalent or multispecific binding agent described herein comprises an Fc fusion of any one of the hPIV-3 HN-specific ISVDs as described herein, or of a humanized variant thereof, such as for instance comprising a sequence selected from any one of SEQ ID NOs: 55-57 or SEQ ID NOs: 60-61.

Further multivalent or multispecific agents are also comprised herein, wherein said ISVD comprised in said multispecific or multivalent agent comprises any one of said humanized variants of the ISVDs disclosed herein, and/or is present in a multispecific or multivalent antibody format known in the art, such as any type of Fc (native or variant IgG, or in particular IgG1 Fcs), or in head-to-tail multivalent or multispecific format, or a combination of head-to-tail and Fc fusion, and/or N- and C-terminally fused ISVDs to an Fc tail; and/or a knob-into-hole Fc fusion format, among others.

In further aspects of the invention, the antigen binding protein as described herein may be labelled, tagged or conjugated. More specifically, a detection label may be useful for localizing, visualizing, and quantitating a binding or recognition event, hence also for in vivo imaging or for diagnostic purposes. The labelled binding agents as described herein can detect HN present on the surface of a virion. Another use for detectably labelled binding agents is a method of bead-based immunocapture comprising conjugating a bead with a fluorescent labelled binding agent and detecting a fluorescence signal upon binding of a ligand. Similar binding detection methodologies utilize the surface plasmon resonance (SPR) effect to measure and detect antigen-binding protein/antigen interactions.

The term detectable label or tag, as used herein, refers to detectable labels or tags allowing the detection and/or quantification of the hPIV-3 HN-specific binding agent as described herein, and is meant to include any labels/tags known in the art for these purposes. Particularly preferred, but not limiting, are affinity tags, such as chitin binding protein (CBP), maltose binding protein (MBP), glutathione-S-transferase (GST), poly(His) (e.g., 6×His or His6), biotin or streptavidin, such as Strep-Tag®, Strep-tag II® and Twin-Strep-Tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase); radioisotopes. Also included are combinations of any of the foregoing labels or tags. Technologies for generating labelled polypeptides and proteins are well known in the art. An antigen-binding protein comprising a hPIV-3 HN-specific ISVD of the invention, coupled to, or further comprising a label or tag allows for instance immune-based detection. Immune-based detection is well known in the art and can be achieved through the application of numerous approaches. These methods are generally based upon the detection of a label or marker, such as described above. See, for example, U.S. Pat. Nos. 3,817,837; 3,850,752; 3,939,350; 3,996,345; 4,277,437; 4,275,149 and 4,366,241. In the case where multiple antibodies are reacted with a single array, each antibody can be labelled with a distinct label or tag for simultaneous detection. Yet another embodiment may comprise the introduction of one or more detectable labels or other signal-generating groups or moieties, or tags, depending on the intended use of the labelled or tagged hPIV-3 HN-specific binding agent of the present invention. Other suitable labels will be clear to the skilled person, and for example include moieties that can be detected using NMR or ESR spectroscopy. Such labelled binding agents, such as hPIV-3 HN-specific ISVDs or Nanobodies as described herein may for example be used for in vitro, in vivo or in situ assays (including immunoassays known per se such as ELISA, RIA, EIA and other “sandwich assays”, etc.) as well as in vivo imaging purposes, depending on the choice of the specific label.

The labelled or tagged binding agents as described herein may also be used as an affinity purification agent. In this process, the labelled agent or antigen-binding protein is immobilized on a solid surface, such as a Sephadex, Sepharose or other polymeric resin, or filter paper, or a cartridge, using methods well known in the art. The immobilized binding agent is subsequently contacted with a sample containing the antigen to be purified, and thereafter the support is washed with a suitable solvent that will remove substantially all the material in the sample except the antigen to be purified, which is bound to the immobilized binding agent. Finally, the support is washed with another suitable solvent, which is capable to outcompete the binding.

In a further aspect of the invention, the antigen-binding protein as described herein may be conjugated to a further functional moiety, such as a therapeutic or half-life extension moiety, or to a cell-penetrant carrier. More specifically, the binders as described herein may as fusion be further coupled or operably linked to further binding moieties, which may be additional ISVDs, or antigen-binding domains specific for a target protein, preferably a target present on the cell surface or extracellularly, or to extend the half-life (e.g. serum albumin specific binders), or alternative compounds that are providing a function. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Another technique for increasing the half-life of a binding agent may comprise the engineering into bifunctional or bispecific domains (for example, one or more ISVDs or active antibody fragments against hPIV-3 HN coupled to one ISVD or active antibody fragment against serum albumin or pulmonary surfactant protein A (Spa) aiding in prolonging half-life)) or into fusions of antibody fragments, in particular immunoglobulin single variable domains, with peptides (for example, a peptide against a serum protein such as albumin). The coupling to additional moieties will result in multispecific binding agent, as further disclosed herein.

In a final aspect of the invention, the antigen-binding protein as described herein which specifically binds hPIV-3 HN, and is at least neutralizing hPIV-3 and Sendai virus, and preferably as well neutralizes hPIV-1, is used as a medicament, or for prevention or treatment of viral infection, specifically parainfluenza viral infection, more specifically human parainfluenza infection. “Human Parainfluenza infection” refers to the invasion of a living subject's tissue by hPIV, multiplication of hPIV, and/or the reaction of host tissues to the infectious hPIV. hPIVs are etiological agents of respiratory infections such as croup, pneumonia, or bronchitis.

In a specific embodiment, said antigen-binding protein also protects against Sendai virus, which may be evaluated for instance by generating a (humanized) mono- or bivalent VHH (as a tandem repeat and/or as an IgG1 Fc fusion), and administer those to mice both prophylactically and therapeutically. As mice are very difficult to infect with hPIV-3 or hPIV-1, the mouse model making use of Sendai virus can indeed be used to get a first in vivo indication of the efficacy, as shown herein. Sendai virus replicates efficiently in DBA/2 mice, which may receive the antigen-binding protein intranasally or intraperitoneally. Morbidity, mortality, viral kinetics and leukocyte infiltration in the lung is then assessed after SeV challenge.

So in a further embodiment the antigen-binding protein as disclosed herein is used for prophylactic and/or therapeutic treatment of a subject against human parainfluenza viral infection, in particular against hPIV-1 and/or hPIV-3 infection. In a specific embodiment, said antigen-binding protein for use in (prophylactic) treatment of a subject against hPIV comprises an ISVD comprising A2R360 or a humanized variant thereof, and/or a multivalent or multispecific agent thereof.

It is to be understood that although particular embodiments, specific configurations as well as materials and/or molecules, have been discussed herein for methods, samples and products according to the disclosure, various changes or modifications in form and detail may be made without departing from the scope of this invention. The following examples are provided to better illustrate particular embodiments, and they should not be considered limiting the application. The application is limited only by the claims.

### Examples

### INTRODUCTION

The present application relates to the identification of VHHs selected from serum from an alpaca immunized with replication competent hPIV-3 virions, and characterized to specifically bind hPIV-3 HN. The VHHs described herein were shown to bind cell surface-expressed hPIV-3 HN at concentrations of 0.01 μg/ml, and neutralized hPIV-3 virions with IC₅₀ values ranging from 197 ng/ml to 6.4 μg/ml. Furthermore, they were found to block HN neuraminidase activity and to compete with the known neuraminidase inhibitor for HN binding, Zanamivir, suggesting a binding epitope near the catalytic site.

Strikingly, for a number of the VHHs cross-reactivity to Sendai virus (SeV) was observed, both in terms of binding and neutralizing capacity, with IC₅₀ values between 2 μg/ml and 11 μg/ml. These VHHs further bound hPIV-1 HN at concentrations of 0.01 μg/ml, even providing for cross-neutralization of hPIV-1 at the highest concentration tested (30 μg/ml). Furthermore, bivalent VHH constructs as a tandem tail-to-head configuration were shown to drastically increase neutralization potency. Thus, for the first time, VHHs with cross-neutralization potency of both hPIV-3 and hPIV-1 were disclosed herein.

### Example 1. HPIV-3 Neutralizing Activity in Alpaca Serum

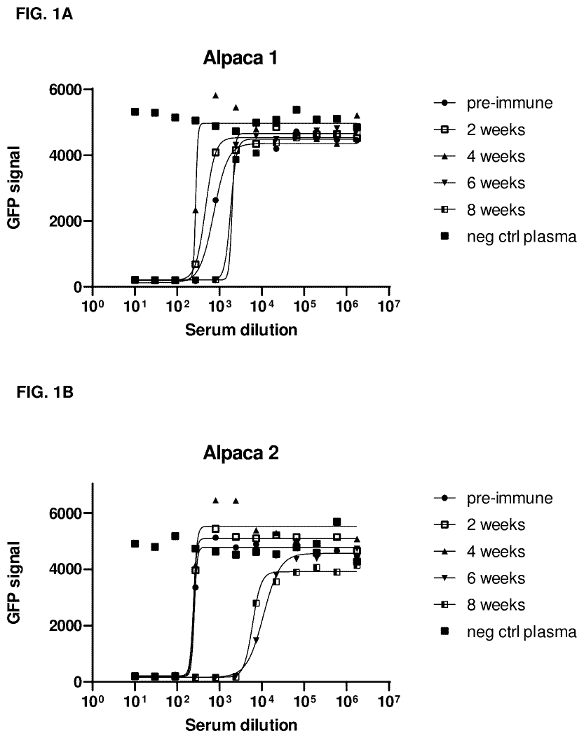

As a first step in generating hPIV-3-specific VHHs, a humoral immune response against hPIV-3 was raised in two alpacas. The animals were first given an intranasal immunization with replication competent hPIV-3 virions, followed by two subcutaneous booster injections containing hPIV-3 and Gerbu adjuvant 27 and 57 days later. Every two weeks during the immunization, blood samples were taken and serum was prepared to monitor the induction of anti-hPIV-3 neutralizing activity (FIGS. 1A and 1B). For this, monolayers of Vero cells were infected with hPIV-3-GFP in the presence of a dilution series of the serum samples. Infection of the cells can be visualized by measuring the GFP fluorescence signal three days post-infection, and a reduction in signal indicates serum-mediated hPIV-3 neutralization. Negative control plasma, being pre-immune plasma from another alpaca, did not show any hPIV-3 neutralizing activity (FIGS. 1A and 1B). In the pre-immune samples of alpacas 1 and 2, some hPIV-3-neutralizing activity could be detected. However, this hPIV-3-neutralizing activity clearly increased at 6 and 8 weeks after immunization around 4-fold for alpaca 1, and 40-fold for alpaca 2, showing that a neutralizing humoral immune response against the virus was raised.

### Example 2. Isolation of hPIV-3-Neutralizing VHHs

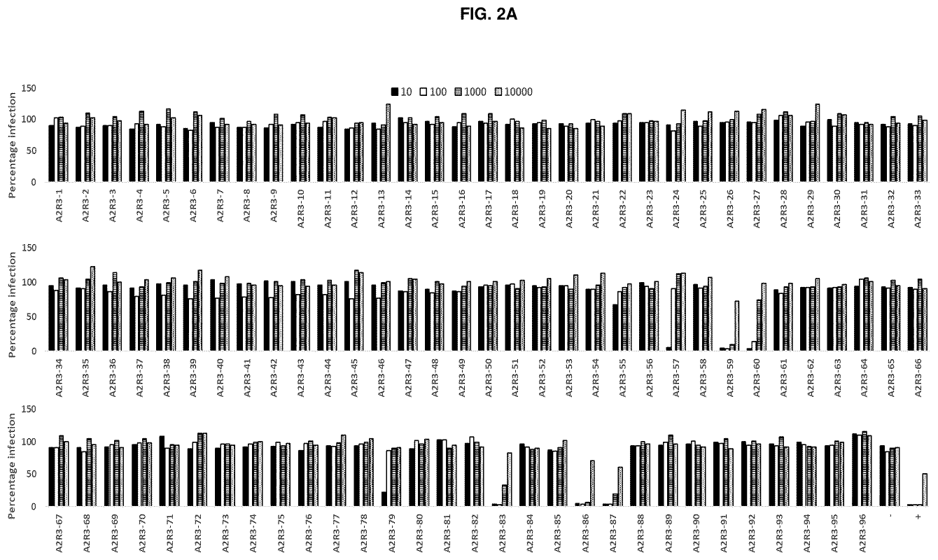

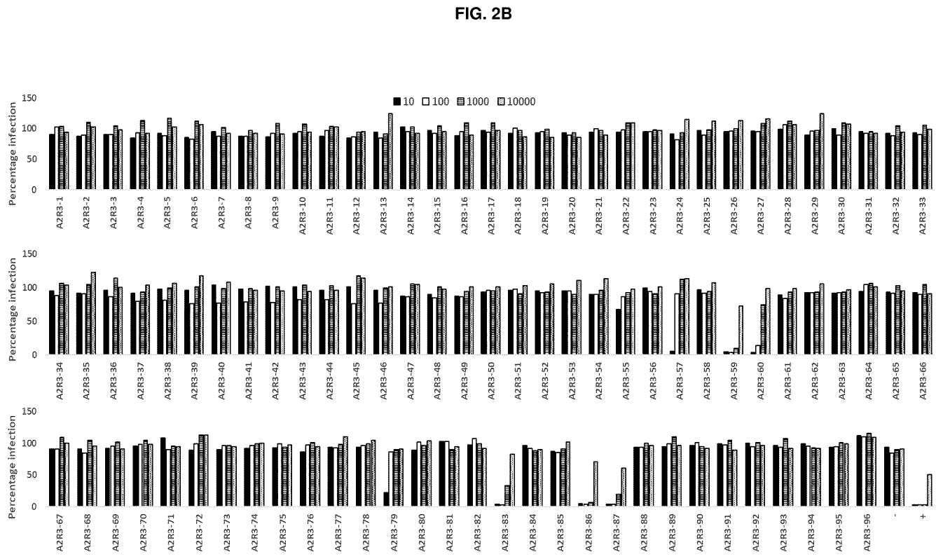

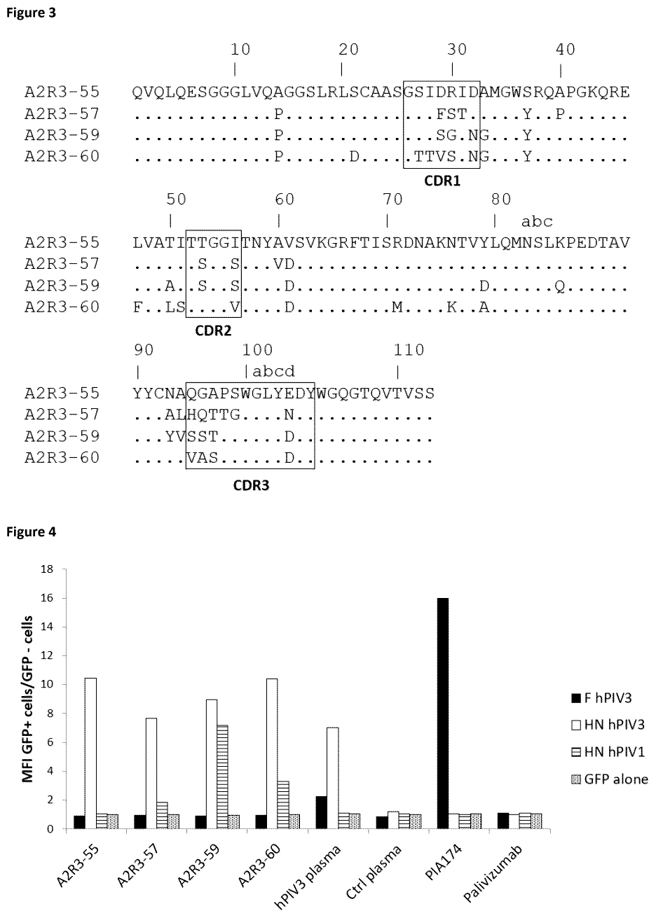

In a second step, we isolated unique hPIV-3 neutralizing VHHs from alpaca 2, which was the best responder. A phage VHH-display library of alpaca 2 was first generated, and subjected to three rounds of panning on immobilized hPIV-3 virions. After the third panning round, 192 clones were randomly selected, and periplasmic extracts were prepared to test for hPIV-3-neutralizing activity. To this end, tenfold dilutions of the periplasmic extracts were added to Vero cells together with hPIV-3-GFP. Of the 192 clones, 20 neutralized hPIV-3-GFP (FIGS. 2A and 2B), with the four preferred clones, belonging to one VHH family, further discussed herein: A2R3-55, A2R3-57, A2R3-59, and A2R3-60 (FIG. 3).

### Example 3. Binding of VHHs to Cells Expressing hPIV-3 and hPIV-1 Hemagglutinin-Neuraminidase (HN)

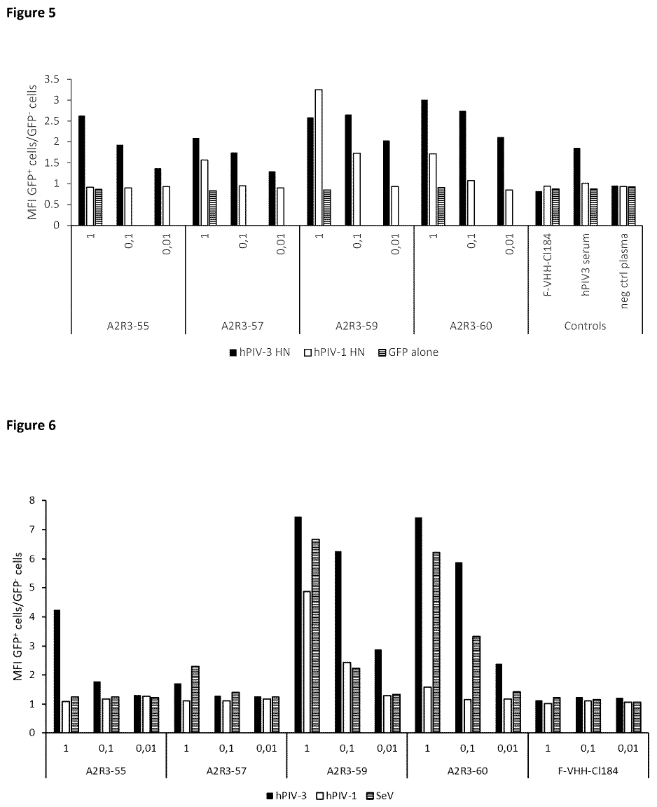

We hypothesized that the hPIV-3-neutralizing VHHs would bind to one of the two hPIV virion envelope proteins that are used for host cell infection, being either the attachment protein hemagglutinin-neuraminidase (HN) or the fusion protein (F). To test this, synthetic VHH sequences of the four clones were cloned into a Pichia pastoris expression vector and subsequently used to transform this yeast. The recombinant VHHs resulting from the yeast cultures were purified from the cell culture media and used for further characterization. Specifically, the binding of the purified recombinant A2R3-55, A2R3-57, A2R3-59, and A2R3-60 VHHs, and of control plasma and antibodies, to hPIV-1 HN-, hPIV-3 HN-, and hPIV-3 F-expressing mammalian cells was assessed by flow cytometry (FIG. 4). hPIV protein expression in mammalian cells was obtained by transfection with codon-optimized expression vectors, combined with a GFP-NLS expression vector; the GFP signal therefore indicates successful transfection. Binding of VHHs, plasma antibodies and pure antibodies to these cells was monitored with AF633-labeled detection antibodies. The results are shown as the AF633 mean fluorescence intensity (MFI) ratio in GFP+(transfected) versus GFP− (non-transfected) mammalian cells. The alpaca hPIV-3 immune plasma sample showed binding to both hPIV-3 F and hPIV-3 HN, indicating the induction of humoral immunity against both targets in the immunized alpaca (FIG. 4). In contrast, all four VHHs clearly bound to hPIV-3 HN, without binding to hPIV-3 F. Strikingly, A2R3-59 and A2R3-60 also showed clear cross-binding activity to hPIV-1 HN. Limited binding to hPIV-1 HN could also be detected for A2R3-57. As the first binding experiment was run at VHH concentrations of 1 μg/ml, we also assessed binding to both hPIV HN proteins at concentrations of 0.1 and 0.01 μg/ml (FIG. 5). All VHHs still bound to hPIV-3 HN, and cross-binding activity of A2R3-59 and A2R3-60 to hPIV-1 HN was also confirmed at these lower concentrations (FIG. 5). Next, we tested binding of the VHHs to Vero cells infected with fluorescently labelled viruses (hPIV-3-GFP, hPIV-1-GFP or Sendai Virus (SeV)-GFP; SeV is the murine hPIV-1 variant) (FIG. 6). All VHHs bound to hPIV-3-infected cells, with varying strengths. Cross-binding to SeV-infected cells could be detected for A2R3-57, A2R3-59 and A2R3-60. Binding to hPIV-1-infected cells could also be detected for A2R3-59, and more limited for A2R3-60. In conclusion, all isolated VHHs are specific for the HN protein, and three of the four monovalent VHHs have cross-binding activity between hPIV-3 HN and hPIV-1 HN.

### Example 4. Determination of IC₅₀ Values for Virus Neutralization of the Purified VHHs

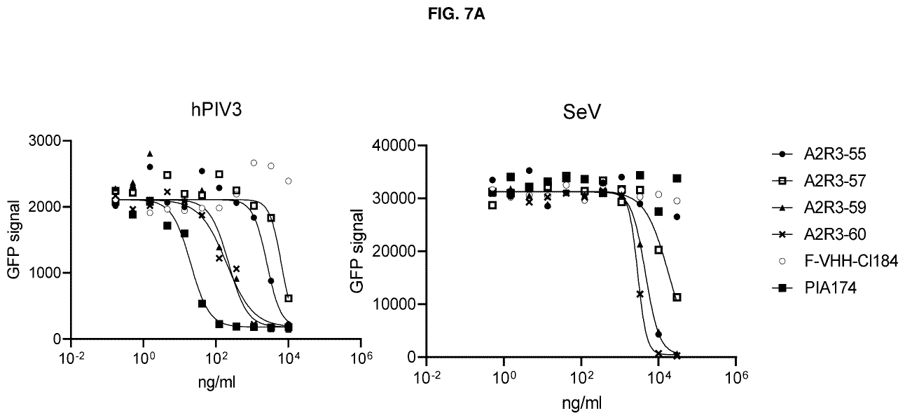

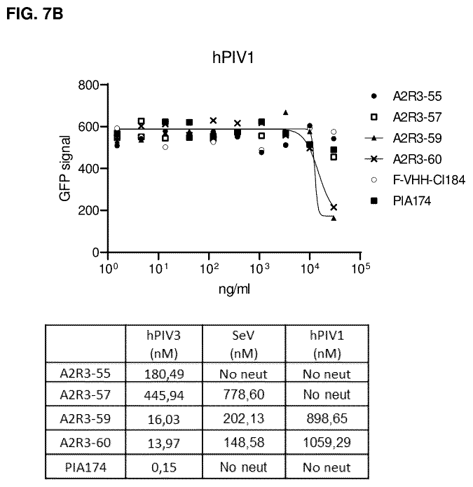

As cross-binding activity was observed for three of the monovalent VHHs, we addressed whether the purified VHHs also had neutralizing activity against hPIV-3-GFP, SeV-GFP and hPIV-1-GFP. For this, the viruses were used to infect Vero cells in the presence of a dilution series of the VHHs (FIGS. 7A and 7B). All four VHHs neutralized hPIV-3-GFP, as evidenced by a reduced GFP fluorescence signal in Vero cells at increasing VHH concentrations (FIGS. 7A and 7B). The IC₅₀ values varied from 14 nM to 446 nM. VHHs A2R3-59 and A2R3-60 also neutralized hPIV-1-GFP at the highest concentration tested (30 μg/ml). SeV-GFP was found to be more susceptible to neutralization by the VHHs than hPIV-1-GFP, with IC₅₀ values between 149 nM and 779 nM for A2R3-57, A2R3-59 and A2R3-60. Thus, in addition to cross-binding activity, the VHH family also has cross-neutralizing activity for hPIV-3 and hPIV-1/SeV.

### Example 5. The Effect of the VHHs on the HN Neuraminidase Activity

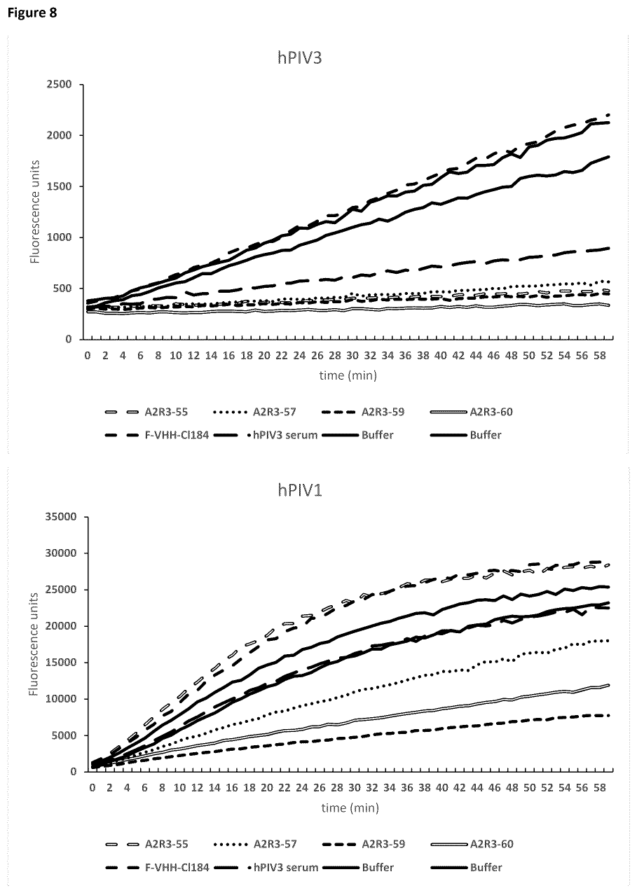

The viral HN protein has both attachment and neuraminidase activities during the host cell infection process. To determine if the VHHs affect the latter, we performed a fluorogenic MUNANA-based assay. VHHs were incubated with hPIV-3 HN- or hPIV-1 HN-expressing cells to which the MUNANA substrate was added (FIG. 8). All four hPIV-3-neutralizing VHHs completely inhibited the hPIV-3 neuraminidase activity. The hPIV-1 HN neuraminidase activity was reduced in the presence of each monovalent A2R3-57, A2R3-59 and A2R3-60 VHH, which are the three cross-binding VHHs.

### Example 6. Zanamivir Competes with the Mono- and Bivalent hPIV-3-Specific VHHs for Binding to hPIV-3 HN

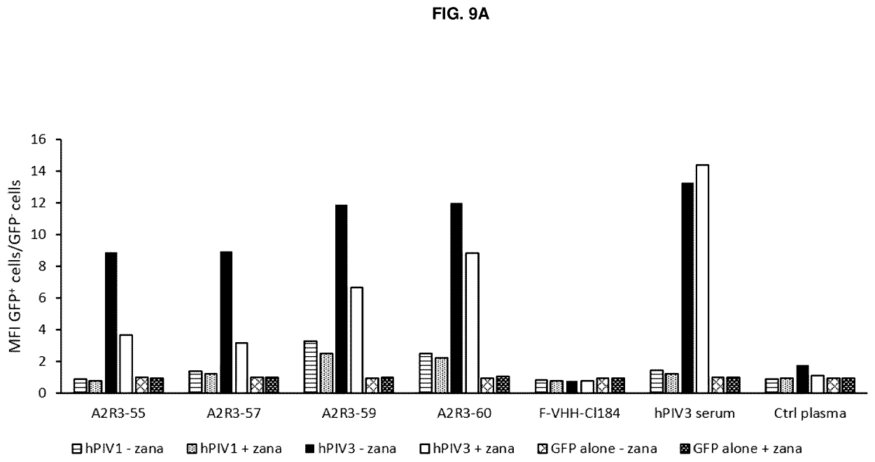

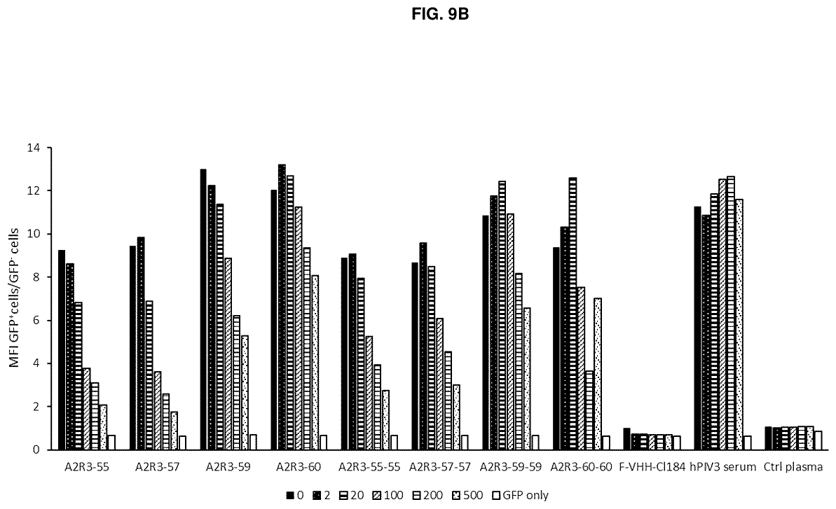

Zanamivir is a small molecule inhibitor of the influenza neuraminidase, which binds to the catalytically active site of this enzyme and also weakly inhibits the hPIV-3 HN activity (23). To determine if the neuraminidase-inhibiting VHHs also bind to an epitope in this region, a flow cytometry-based competition assay was performed. We analyzed monovalent VHH binding to hPIV-1 HN and hPIV-3 HN as expressed on the cell surface of mammalian cells, in the presence or absence of zanamivir (FIG. 9A). The AF633 MFI is a measure for VHH binding, whereas the GFP signal identifies cells transfected with hPIV HN. Results are shown as the AF633 MFI ratio of GFP+ versus GFP− mammalian cells. Binding to hPIV-3 HN was clearly reduced in the presence of zanamivir for all four VHHs, showing competition of the VHHs with Zanamivir (FIG. 9A). This was not the case for the hPIV-3 immune serum of alpaca 2. Binding of A2R3-59 and A2R3-60, the strongest hPIV-1 binders, to hPIV-1 HN was only slightly reduced in the presence of Zanamivir, suggesting a less pronounced competition. The competition assay was repeated for binding to hPIV-3 HN using a dilution series of zanamivir (FIG. 9B). Increasing concentrations of zanamivir reduced the binding of the VHHs to hPIV-3 HN. At 500 μg/ml of zanamivir, binding of A2R3-55 and A2R3-57 was reduced approximately 5-fold, while this was only 2.4-fold and 1.5-fold for VHHs A2R3-59 and A2R3-60 respectively. Bivalent VHHs were generated, by genetically linking VHHs with a 15 AA (G4S)3-linker. The bivalent VHHs behaved similarly as their monovalent counterparts, suggesting that the VHHs bind to an epitope in HN that comprises (part of) the catalytic site (FIG. 9B).

### Example 7. Bivalent VHHs Display Increased Cross-Neutralizing Activity Against hPIV-3, hPIV-1 and SeV

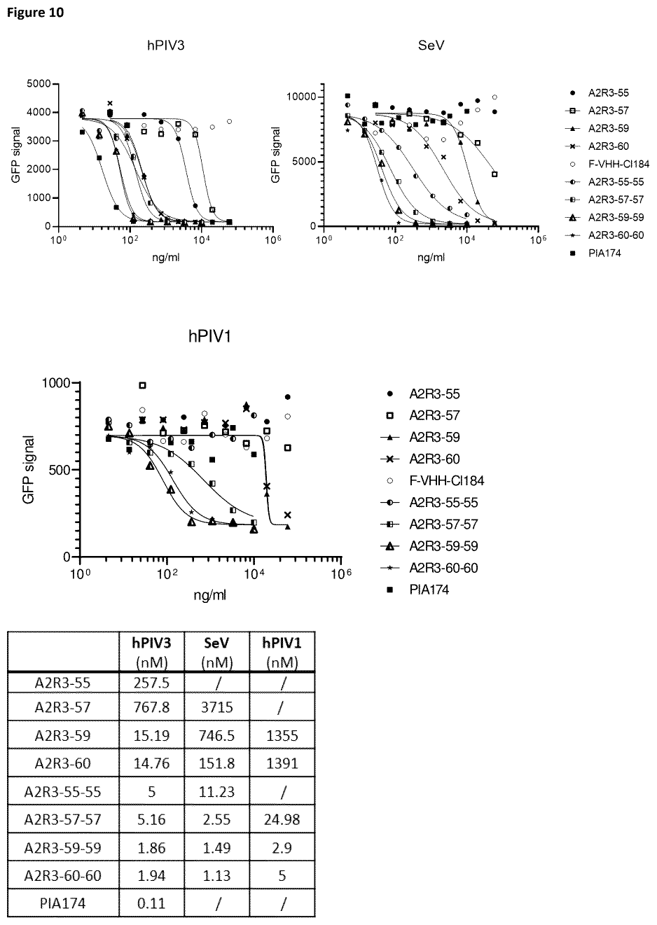

The valency of a VHH-antigen interaction can be increased by generating tandem tail-to-head fusion constructs in which two VHH copies are genetically linked to each other, separated by a flexible linker sequence as used herein for instance: GGGGSGGGGSGGGGS (SEQ ID NO:46). We generated such bivalent construct sequences for A2R3-55, A2R3-57, A2R3-59, and A2R3-60, expressed them in Pichia pastoris, and purified the resulting bivalent VHHs from the culture media. We then tested their potency of neutralizing GFP-expressing viruses (FIG. 10). Indeed, all four bivalent VHHs neutralized hPIV-3-GFP with a significantly higher potency than their monovalent counterparts: approximately 50-, 150-, 8- and 7.5-fold for A2R3-55, A2R3-57, A2R3-59 and A2R3-60, respectively. The bivalency resulted in higher cross-neutralizing activity. All four bivalent VHHs neutralized SeV, with IC₅₀ values between 1.13 and 11.23 nM. Bivalent A2R3-57-57, A2R3-59-59, and A2R3-60-60 also neutralized hPIV-1, with A2R3-59-59 being the most potent (IC₅₀ value of 2.9 nM).

### Example 8. Amino Acid Residues D275 and R277 in hPIV-3 HN are Involved in VHH Binding to hPIV-3

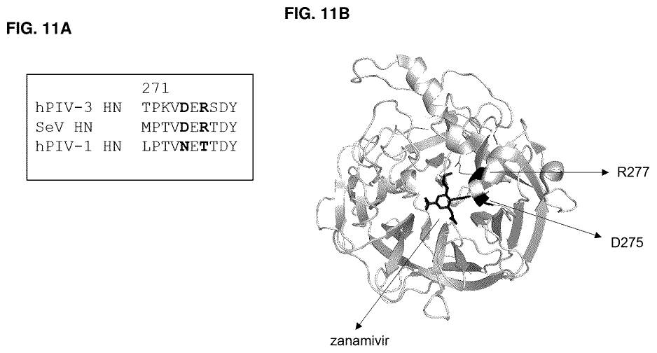

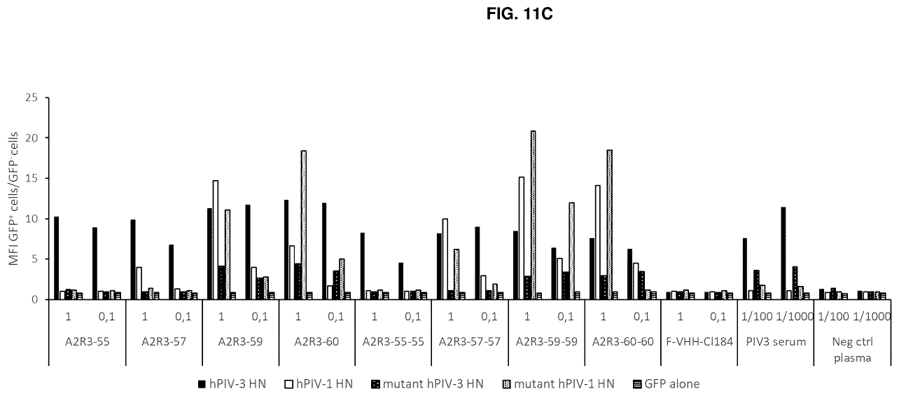

Remarkably, SeV is more susceptible to neutralization by the VHHs than the human PIV-1. To learn more about the VHH binding epitopes, we aligned representative hPIV-1, SeV, and hPIV-3 HN amino acid sequences with a focus on the hPIV-3 HN amino acids 271 to 280, which represent a small part of a region near the catalytic site (FIG. 11A). In this alignment, two residues near the active site of hPIV-3 HN (D275 and R277) were found to be conserved in SeV HN, yet different in hPIV-1 HN. To analyze if these residues are involved in the binding of the VHHs to HN, expression vectors for hPIV-1 and hPIV-3 HN point mutants were generated. Specifically, we introduced two mutations (D275N and R277T) in hPIV-3 HN. We noticed a loss of binding of mono- and bivalent VHHs A2R3-55 and A2R3-57 (which do not, or very poorly bind to hPIV-1 HN), but not for the mono- and bivalent VHHs A2R3-59 and A2R3-60 (FIG. 11C). Reciprocally, introduction of N to D and T to R mutations at the corresponding positions in hPIV-1 HN was associated with enhanced binding of monovalent VHH A2R3-60 (which binds SeV-infected cells better than wild type hPIV-1-infected cells), and of bivalent VHHs A2R3-59-59 and A2R3-60-60 (FIG. 11C). This shows that the difference in reactivity against hPIV-1 HN and SeV HN can indeed be linked to these two residues (D275 and R277 in hPIV-3 HN) which are conserved in SeV HN. These two residues are thus probably part of the epitope of the four VHHs.

### Example 9. Design and Generation of VHH Variant Sequences for In Vivo Applications

To minimize the immunogenicity risk, framework regions of the bivalent constructs were altered to obtain a more humanized VHH sequence, based on sequence alignment with hlGHv3, according to known practice as described herein, and as illustrated herein for VHH 2AR3-60, -59, and -57. Those humanized VHH variants illustrated herein having specifically the following substitutions:
for VHH 2AR3-60: Q1D/E, Q5V, D21S, M71R, K76N, A79Y, K83R, Q108L, as exemplified in SEQ ID NO: 64, 67, 52,
for VHH 2AR3-59: Q1D/E, Q5V, D79Y, Q83R, Q108L as exemplified in SEQ ID NO:65, 68, 53,
for VHH 2AR-57: Q1D/E, Q5V, P40A, V60A, K83R, Q108L as exemplified in SEQ ID NO:66, 69, 54.

The bivalent (head to tail humanized bivalent ‘version 1’) constructs (SEQ ID NOs:52-54) have been produced and are characterized for their binding and neutralization capacity, in line with the monovalent and bivalent wild-type sequences.

In addition, the humanized VHH variants were fused to a human IgG1 Fc, as shown in SEQ ID NOs: 55-57, resp., or which may be provided with a 1E substation as shown in SEQ ID NOs:67-69 for the VHHs, in a fusion with an Fc. To rule out Fc-mediated effector functions, LALA-PG mutations were introduced (Schlothauer, et al. 2016, Protein Eng. Des. Sel. PEDS 29, 457-466) in the exemplified sequences, though Fc sequences from native IgG1 or IgG4, as well as other variants known in the art and used therapeutically, may as well be applied for fusion with the VHHs. In addition, two further humanized variant constructs were designed for the bivalent A2R3-59 and -60 VHHs, wherein the original Gly at position 33 has been substituted by an Ala as to remove a possible deamidation risk on Asn at position 32, in bivalent head to tail format, as well as Fc fusion (SEQ ID NOs: 58-61).

### Example 10: Humanization does not Affect Neutralizing Activity

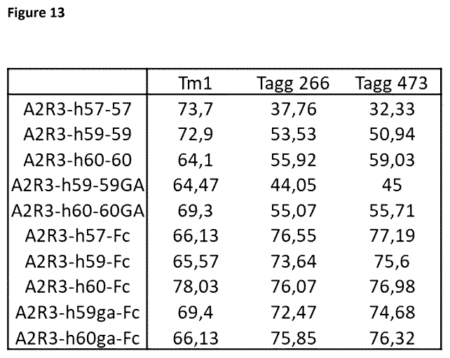

The stability of the humanized bivalent VHHs and humanized VHH-Fc fusions was analyzed in an Uncle platform (FIG. 13). This analysis showed that the antibody with the A2R3-h60-Fc sequence (SEQ ID NO: 55) is the most optimal molecule in view of thermal stability, with a thermal melt temperature (Tm) of 78.03° C. and aggregation temperature (Tagg) of 76.07° C. (measured at OD₂₆₆) and 76.98° C. (measured at OD₄₇₃).

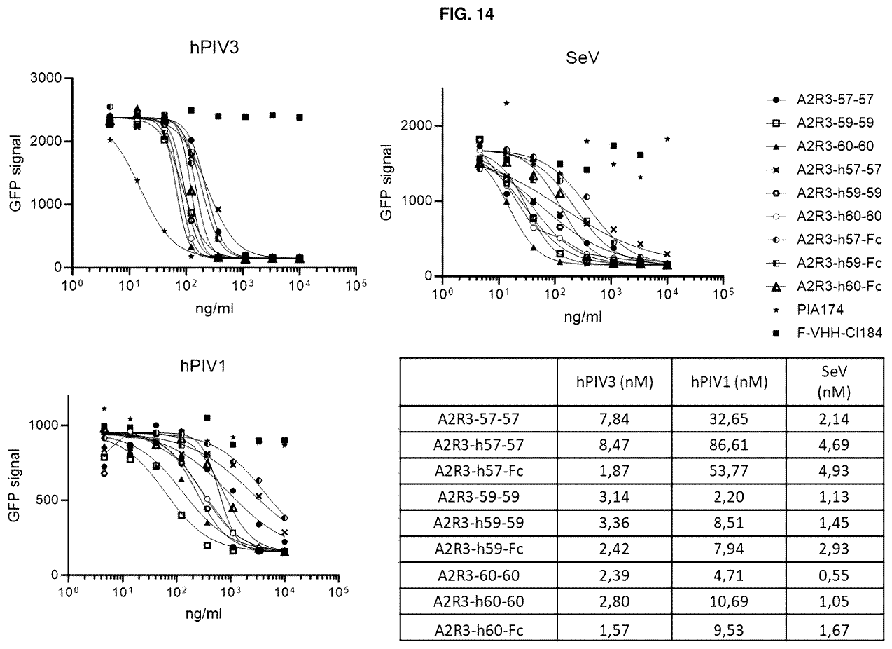

To determine if humanization the VHHs has an impact on the antiviral activity of the VHHs, the humanized bivalent (head-to-tail) VHHs and the humanized bivalent VHH-Fc antibodies (recombinant expression of SEQ ID NOs: 52-57) were tested in neutralization assays against GFP-expressing viruses (FIG. 14). The humanized bivalent constructs have IC₅₀s in a similar range as the wild-type bivalent constructs, indicating that the antiviral potency of the humanized constructs was not significantly affected. Also fusion of the humanized VHHs to a human IgG1-derived Fc-domain had only a minimal effect on the neutralization capacity and, in some occasions, was associated with increased potency (e.g. for h57-Fc neutralizing hPIV-3) and sometimes a small negative impact (e.g. for h59-Fc neutralizing hPIV-1). So humanization variants did not significantly alter the neutralization potency of the VHH constructs.

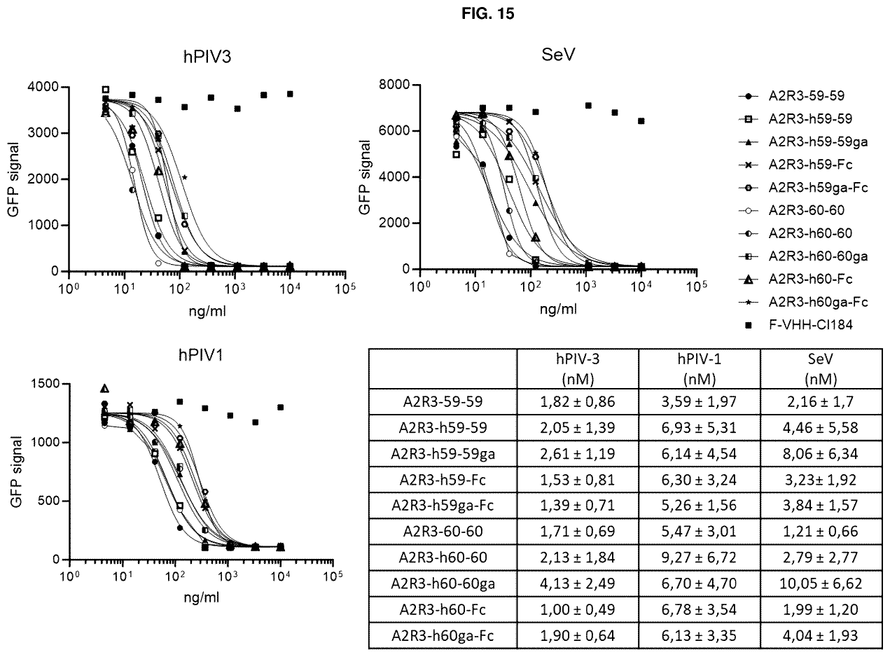

Next, we evaluated if the G33A substitution, which is close to CDR1, in A2R3-h59 and A2R3-h60 had an effect on the neutralizing activity (SEQ ID NOs: 58-61) (FIG. 15). This substitution did not impact neutralization of hPIV-3 and hPIV-1, and was associated with a two- to four-fold drop in neutralizing activity against SeV compared to the wild-type humanized variant.

### Example 11. Cross-Binding to hPIV-4a and -4b

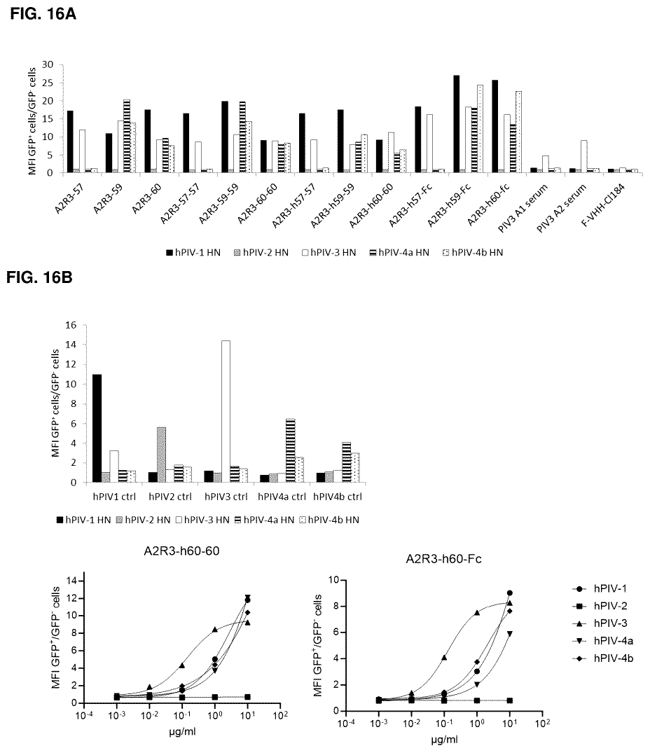

Hemagglutinin-neuraminidase (HN) of hPIV-1 and -3 (belonging to the Respirovirus genus) share only 25-29% amino acid sequence identity with the HN of hPIV-2 and -4 (belonging to the Rubulavirus genus). Still several residues in the active site of the protein are conserved, which prompted us to test possible cross-binding to hPIV-2, -4a and -4b HN. Binding of the VHHs and VHH-Fcs to hPIV-2, -4a and -4b HN was tested by flow cytometry. For this assay HEK293T cells were transfected with a GFP-NLS-expression vector together with an expression vector containing a codon-optimized version of a hPIV-1, -2, -3, -4a and -4b HN gene (FIG. 16). VHH A2R3-59 and -60 could bind to hPIV-4a and -4b HN next to the expected binding to hPIV-1 and -3. No binding could be detected to hPIV-2 HN, even though, control sera (upper panel in FIG. 16B) did show binding to the respective virus showing that hPIV-2 was bound by the control serum for hPIV-2. Also, their derived constructs (bivalent, humanized bivalent and VHH-Fc) have the same binding profile. A2R3-h60-60 and A2R3-h60-Fc have the same binding affinity to hPIV-4a and -4b HN as to hPIV-1 HN (FIG. 16). Despite this cross HN-binding activity, neutralization of hPIV-4a and -4b by A2R3-h60-60 or A2R3-h60-Fc could not be demonstrated.

### Example 12. A2R3-h60-Fc Antibodies Protect Against SeV Infection In Vivo

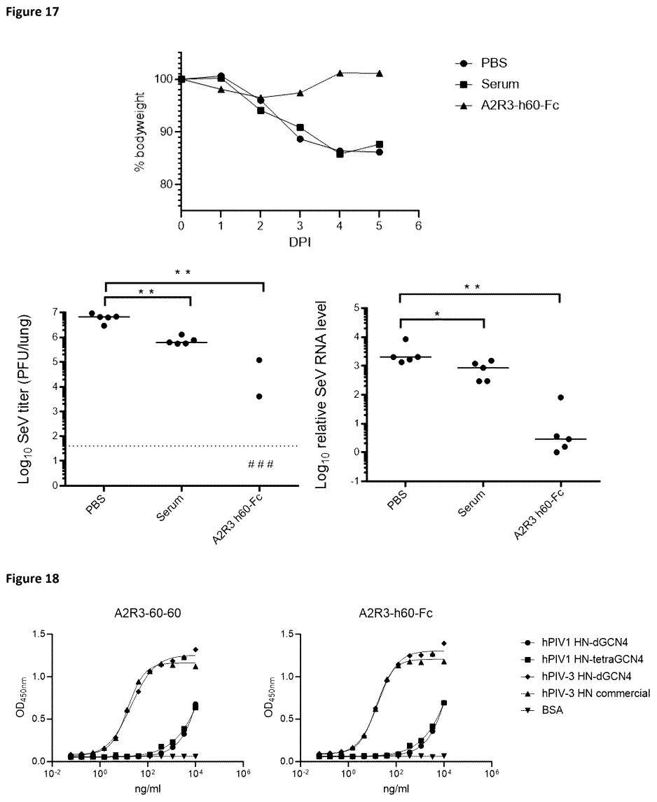

Recombinantly produced A2R3-h60-Fc antibody was selected for an in vivo challenge study because of its potent neutralizing activity and favorable stability profile. A prophylactic set-up was tested in which 100 μg (corresponding to 5 mg/kg) of A2R3-h60-Fc antibody was injected intraperitoneally to DBA/2J mice 24 hours before inoculation with 1000 PFU of SeV/52. Controls consist of a PBS group (negative control) and a immune serum-treated group (positive control). Mice were weighed daily and five days after challenge the mice were sacrificed and viral lung titers were determined (FIG. 17). A2R3-h60-Fc antibody administration protected completely against weight loss, whereas neither of the control groups were protected. For three of the five mice in the A2R3-h60-Fc antibody group, no virus could be detected in the lungs based on a plaque assay. The two remaining mice had SeV titers that were 50- to 1500-fold lower than the PBS group. This significant reduction in lung virus load in the A2R3-h60-Fc antibody treated mice could be explained, at least in part, due to an in vitro effect of the remaining VHH-Fc antibody in the lung homogenates. Therefore, viral RNA was also quantified with a SeV-specific RT-qPCR. In mice treated with the A2R3-h60-Fc antibody 690-times less viral RNA could be detected compared to the negative control group, based on median values. The serum treatment was less effective than the A2R3-h60-Fc antibody treatment and led to a smaller reduction in viral titers and could not protect against weight loss upon infection.

### Example 13. Production and Analysis of Recombinant hPIV-1 and -3 HN Protein

Recombinant hPIV-1 and hPIV-3 HN were produced for further characterization of the VHHs. Hereto, the head domain of hPIV-1 and hPIV-3 HN were N-terminally linked to a oligomerization domain, either dimeric GCN4 (dGCN4) or tetrameric GCN4 (tetraGCN4) preceded by a strep-tag II. Constructs were produced in HEK293s cells and purified using their strep-tag II. HPIV-3 HN linked to tetraGCN4 was poorly produced and was omitted from further analysis. The remaining purified HN proteins were active in a neuraminidase activity assay. A2R3-60-60 and A2R3-h60-Fc could bind to these recombinant proteins with ECsos of respectively 167 and 32 μg/ml for hPIV-1 HN-dGCN4, 50 and 71 μg/ml for hPIV-1 HN-tetraGCN4 and 20 and 18 ng/ml for hPIV-3 HN-dGCN4 (FIG. 18). The two VHH-constructs bind hPIV-3 HN-dGCN4 as efficiently as a commercially available hPIV-3 HN protein.

### Materials and Methods

Immunization and phage VHH-display library generation. The alpaca immunizations were performed in a biosafety level 2 contained stable at the Faculty of Veterinary Sciences of Ghent University following a protocol that was approved by the ethical committee of the faculties of veterinary medicine and bioscience engineering of Ghent University (application number 2019-84). Two alpacas were intranasally (i.n.) immunized with 6*10⁸ PFU of hPIV-3 (NIH 47885, Bei Resources) on day 0, followed by two subcutaneous boosts with 2*10⁸ PFU of hPIV-3 on days 27 and 57. The intranasal administration was performed without adjuvant whereas the two subcutaneous injections were adjuvanted with Adjuvant P (Gerbu). Blood samples were taken every two weeks by puncture of the jugular vein and serum was prepared. On day 62, 100 ml of anticoagulated blood was collected for the isolation of lymphocytes.

Total RNA from peripheral blood lymphocytes was prepared with the RNeasy Midi Kit (Qiagen), and the resulting RNA was used as template for first strand cDNA synthesis with oligo-dT primers. Using this cDNA, the VHH-encoding sequences were amplified by PCR using a primer set (SEQ ID NOs: 62-63) including Sapl sites, and cloned into the Sapl site of the phagemid vector pMECS-GG. Electrocompetent E. coli TG1 cells were transformed with the recombinant pMECS-GG vector resulting in one independent VHH library per alpaca. Each library consisted of about 10′ independent transformants. For both libraries, about 90% of the transformants harbored the vector with the right insert size, as evidenced by PCR analysis of randomly selected independent transformants. VHH-presenting phages, for use in bio-panning, were produced by infecting 300 μl of the library stock with VCS M13 helper phage.

Cells. Vero cells (ATCC, CCL-81), Vero (E6) TMPRSS2 cells (NIBIOHN, JCRB1819), LLC-MK2 Derivative cells (IRC cell bank) and HEK-293T cells (a gift from Dr M. Hall, University of Birmingham) were grown in DMEM medium containing high glucose, GlutaMAX™ Supplement, and pyruvate (Gibco, 31966021) and supplemented with 10% heat-inactivated fetal calf serum (FCS) and non-essential amino acids (Invitrogen, Carlsbad, California). Vero (E6) TMPRSS2 cells were cultured in the presence of Geneticin (G418) (1 mg/ml), yet this was not added when cells were seeded for assays.

Viruses. hPIV-3 (NIH 47885) was obtained from the BEI Resources repository. GFP-expressing hPIV-3 (hPIV-3-GFP) and GFP-expressing hPIV-1 (hPIV-1-GFP) were obtained from ViraTree. GFP-expressing Sendai Virus (SeV) (SeV-GFP) was kindly provided by Valery Z. Grdzelishvili (University of North Carolina at Charlotte). These viruses were propagated by infecting monolayers of LLC-MK2 cells with 0.1 MOI of virus in the presence of growth medium without fetal calf serum (and with 2 μg/ml trypsin added for hPIV-1-GFP). Three days after infection, the cells and growth medium were collected and clarified by centrifugation. Clarified medium was aliquoted, snap-frozen in liquid nitrogen and stored at −80° C.

Sev/52 was obtained from ATCC (ATCC-VR-105). This virus was propagated by inoculating embryonated chicken eggs. In brief, 10-day old fertilized chicken eggs were inoculated by injection in the allantoic cavity of 10 μl stock virus. After two days incubation at 37° C. the eggs were incubated overnight at 4 degrees Celsius, after which the allantoic fluid was harvested. The fluid was cleared from debris by centrifugation at 1800 g for 10 minutes, and the resulting supernatant was aliquoted, snap-frozen in liquid nitrogen and transferred to −80° C. for long-term storage.

hPIV-1, hPIV-3 and SeV neutralization assays. Alpaca serum samples, periplasmic extracts, and purified VHHs were tested for neutralizing activity against hPIV-3-GFP. For the alpaca serum samples, Vero cells were seeded in a 96-well plate (15,000 cells/well). The next day, a dilution series of the serum samples was prepared in Fluorobrite™ DMEM culture medium, containing 2 mM L-glutamine, non-essential amino acids, 1 mM sodium pyruvate, 1% penicillin and 1% streptomycin (threefold dilution series, starting from a tenfold dilution). An equal volume of hPIV-3-GFP suspension (diluted to 100 PFU/μl) was added to the serum dilutions, followed by incubation for 30 minutes at 37° C. Subsequently, 50 μl of the serum/hPIV-3-GFP suspensions was added to the Vero cells, which had been washed with the Fluorobrite™ DMEM culture medium. After two hours of incubation, another 50 μl of Fluorobrite™ DMEM culture medium was added to every well. Three days post infection, the GFP signal was measured using a Tecan Infinite 200PRO M-Plex plate reader.

The same method was used to determine neutralizing activity of periplasmic extracts (obtained as explained below). Tenfold dilution series of samples, starting from a tenfold dilution, were prepared. Alpaca hPIV-3 immune plasma was used as a positive control, and a periplasmic extract from a clone producing VHHs specific for irrelevant SARS-Cov2 receptor binding domain was used as a negative control.

To determine the IC₅₀ of purified monovalent or bivalent VHHs, threefold serial dilutions of these VHHs were prepared in Fluorobrite™ DMEM culture medium and evaluated for hPIV-3-GFP-neutralizing activity using the same method, though, with some different parameters: in Example 4 (FIGS. 7A and 7B), Vero (E6) TMPRSS2 cells were used for hPIV-1, while Vero cells were used for hPIV-3 and SeV assays, and the incubation period after infection was respectively 2 and 3 days; whereas in Example 7 (FIG. 10), Vero (E6) TMPRSS2 cells were used for all 3 viruses, and with an incubation period of 2 days after infection, and hPIV-1-GFP and SeV-GFP neutralization assays were performed in a similar way. IC₅₀ values were calculated using the Graphpad Prism 8 software.

Isolation of hPIV-3-neutralizing VHHs. To enrich for hPIV-3-binding phages in the library derived from alpaca 2, three rounds of panning on hPIV-3-coated wells were performed. One well (well A1) of a microtiter plate (F96 MaxiSorp, Nunc) was coated overnight with 100 Ip of hPIV-3 viral stock (for the 1^{st} round a stock of 3.7*10⁸ PFU/ml, and for the 2^{nd} and 3^{rd} rounds a stock of 7.8*10⁶ PFU/ml was used). Another well (well A12) was coated with uninfected LLC-MK2 cell supernatants and served as a negative control well. The two wells were blocked during one hour with 4% milk, Pierce Protein-Free blocking buffer, and SEA BLOCK blocking buffer for the 1^{st}, 2^{nd} and 3^{rd} round respectively. Next, 10¹¹ phages in a volume of 100 μl of the same blocking buffer were added to these two wells. After one hour, the unbound phage particles were removed and the wells were washed ten times with PBS. The retained phages were then eluted by applying 100 μl alkaline TEA-solution (14% triethylamine (Sigma) pH 10) to the wells for exactly ten minutes. The dissociated phages were then transferred to a sterile tube with 100 μl 1M TRIS-HCl pH 8.0. Tenfold serial dilutions of the eluted phages were prepared in PBS, and 10 μl of this dilution series was used to infect 90 μl of TG1 cells (phage display competent E. coli cells). Infection was allowed for 30 minutes at 37° C., after which the bacteria were plated on LB/agar plates with 100 μg/ml ampicillin and 1% glucose. The enrichment for hPIV-3-antigen-specific phages by this panning procedure was assessed by comparing the number of phagemid particles eluted from the hPIV-3-coated well with the number of phagemid particles eluted from the negative control well. The remaining volume of the eluted phages was used to infect TG1 cells which were also infected with M13VCS helper phages. These cells were transferred to a shake flask containing 300 ml 2×TY with ampicillin and kanamycin. After overnight incubation at 37° C., the VHH-displaying phages were precipitated from the bacterial supernatant and a next round of panning was performed.

After panning, 192 ampicillin-resistant colonies were randomly selected for further analysis. These colonies were first transferred to a fresh LB/agar plate with ampicillin and then used to inoculate 1 ml of Terrific Broth (TB) medium with 100 μg/ml ampicillin in 24 deep-well plates. Inoculated plates were incubated at 37° C. for 5 hours while shaking. VHH expression was induced by adding isopropyl β-D-1-thiogalactopyranoside (IPTG) until a concentration of 1 mM. The plates were subsequently incubated overnight at 37° C. while shaking. The next day, bacterial cells were pelleted by centrifugation (12 minutes at 3200 rpm) and the supernatants were removed. The cell pellet was resuspended in 200 μl TES buffer (0.2 M Tris-HCl pH 8, 0.5 mM EDTA, 0.5 M sucrose) and the plates were shaken at 4° C. for 30 minutes. Next, water was added to the resuspended cells to induce an osmotic shock, leading to the release of the periplasmic proteins including the VHHs. The deep-well plates were incubated at 4° C. while shaking for 1 hour, followed by centrifugation, and the supernatants, containing the periplasmic extracts, were recovered.

The periplasmic extracts were tested in a hPIV-3-GFP neutralization assay as described above. All clones which showed neutralizing activity were selected for further analysis. The corresponding bacteria were grown in 2 ml of LB medium with ampicillin selection for plasmid isolation using the Wizard Plus SV miniprep DNA Purification System (Promega). The cDNA sequences of the cloned VHHs were determined by Sanger sequencing using the MP075 primer as in SEQ ID NO: 47.

Cloning of VHHs into Pichia pastoris expression vector and transformation of Pichia pastoris. Synthetically produced nucleotide sequence molecules were ordered for five unique monovalent VHHs, as well as for four bivalent constructs consisting of two identical VHHs linked by a (G4S)3 linker (gBlocks gene fragments, IDT). Internal amber stop codons, N-glycosylation sites and BsmBI sites were removed from the codon-optimized sequences, and overhangs with BsaI and BsmBI sites were added. The sequences were transferred to a Pichia pastoris expression vector using a modular cloning technique based on type IIs restriction enzymes as described in Lee et al., 2015 (17). In the expression vector, the VHH sequences are under the control of the methanol-inducible AOX1 promoter. The vector further codes for an 8×His tag, which will be linked to the resulting VHHs, the pre-pro secretion signal of the S. cerevisiae alpha mating factor to direct secretion of the resulting VHHs, and a Zeocine resistance marker for selection in bacterial as well as in yeast cells. The vectors were linearized in the AOX1 promoter (with Pmel) before transformation to Pichia pastoris to promote homologous recombination in the endogenous AOX1 locus for stable integration into the genome. The condensed transformation protocol, as described by Lin-Cereghino et al., 2005 (18), was used to transform Pichia pastoris strain GS115.

Purification of VHHs produced by Pichia pastoris. Expression of VHHs by transformed Pichia pastoris clones was first analyzed in 2 ml cultures. On day 1, individual transformants were used to inoculate 2 ml of YPNG medium (2% pepton, 1% Bacto yeast extract, 1.34% YNB, 0.1 M potassium phosphate pH 6, 1% glycerol) containing 100 μg/ml Zeocin (Life Technologies), followed by incubation at 28° C. for 24 h while shaking. The next day, cells were pelleted by centrifugation (8 minutes at 500×g), the YPNG medium was replaced by YPNM medium (2% pepton, 1% Bacto yeast extract, 1.34% YNB, 0.1 M potassium phosphate pH 6.1% methanol) to induce VHH expression, and cultures were incubated at 28° C. for 72 h while shaking. 72, 80 and 96 h after transfer to YPNM medium, 50 μl of 50% methanol was added to the cultures, and 4 h after the last addition, the yeast cells were pelleted by centrifugation (8 minutes at 500×g) and the supernatants were retained to assess the presence of VHHs. Crude medium (25 μl) was loaded on a 15% SDS-PAGE gel, after which presence of protein was analyzed by Coomassie Brilliant Blue staining.

Pichia pastoris transformants that yielded high levels of VHHs in the medium were selected for scale-up using 100 ml Pichia cultures. Growth, methanol-induced expression of VHHs, and medium harvesting were similar as mentioned above for the 2 ml cultures. The cleared culture medium was loaded on a 1 ml HisTrap HP column (Cytiva) which was pre-equilibrated with the HisTrap binding buffer (20 mM sodium phosphate, 0.5 M NaCl, 20 mM imidazole, pH 7.4). After washing the column with 10 column volumes of HisTrap binding buffer, the bound proteins were eluted with a linear imidazole gradient starting from 20 mM and ending at 500 mM imidazole in binding buffer. Fractions containing the VHHs, as determined by SDS-PAGE analysis, were pooled, buffer-exchanged to PBS and concentrated with a Vivaspin column (5 kDa cutoff, Sartorius).

Binding of VHHs to HEK293T cells expressing hPIV proteins. The binding of the purified VHHs to hPIV-3 F, hPIV-3 HN, or hPIV-1 HN proteins expressed on the surface of cells transfected with a hPIV-3 F, hPIV-3 HN, or hPIV-1 HN expression vector was evaluated by flow cytometry. HEK293T cells were seeded at 2*10⁶ cells per 100 mm tissue culture plate and transfected with 2 μg of pCAXL-hPIV-3-F, -hPIV-3-HN, or -hPIV-1-HN, which codes for codon-optimized hPIV-3 F, hPIV-3 HN, or hPIV-1 HN, respectively, using PEI transfection reagent. To trace transfected cells, transfections were performed in the presence of 1.5 μg of peGFP-NLS. Control transfections were performed with an empty pCAXLvector in combination with peGFP-NLS. One day after transfection, the cells were detached and incubated for 30 minutes in PBS containing 1% bovine serum albumin (BSA) (PBS/BSA). Subsequently, the cells were incubated with the indicated VHHs or antibodies (at 1 μg/ml or indicated concentrations), or alpaca plasma or serum (1/50 dilution). One hour later, the cells were washed once with PBS/BSA and incubated with mouse anti-histidine-tag antibodies (Biorad, MCA1396) for VHH samples, goat anti-llama antibodies (ImTec diagnostics) for alpaca plasma or serum samples, and PBS/BSA for antibody samples for 1 hour. Next, the cells were washed once with PBS/BSA and anti-mouse-IgG-Alexa633 antibodies (for VHH samples), anti-goat-Alexa633 antibodies (for alpaca plasma and serum samples) and anti-human-Alexa633 antibodies (for antibody samples) were added during 30 minutes combined with the eBioscience Fixable Viability Dye eFluor 506 (Invitrogen). After washing the cells with PBS/BSA, the cells were resuspended in PBS and analyzed using a BD® LSR II Flow Cytometer. Living cells were gated and the median fluorescence intensity (MFI) of the AF633 signal was measured for both the GFP⁺ cells and GFP-cells.

Binding to infected cells. Vero (E6) TMPRSS2 cells were seeded at 5*10⁶ cells per T75 cell culture flask one day before infection with 1 MOI of hPIV-3-GFP, hPIV-1-GFP or SeV-GFP. One day after infection, the cells were trypsinized, blocked with BSA and stained with 1 μg/ml, 0.1 μg/ml or 0.01 μg/ml VHHs. The cells were fixed with 1% paraformaldehyde (PFA) overnight and washed with PBS/BSA before further staining and analysis as described in the paragraph above.

Neuraminidase inhibition assay. HEK293T cells were seeded at 1×10⁶ cells in 100 mm tissue culture dishes two days before transfection with an hPIV-3 or hPIV-1 HN expression vector as described above. One day after transfection, the cells were washed with PBS, loosened with PBS and resuspended in McIlvain buffer at pH 5.4. 3×10⁵ cells per reaction were then mixed with VHHs (5 μg/ml) or alpaca hPIV-3 immune serum (1/20 dilution) and an end volume of 40 pL was added to a black 96-well OptiPlate. Just before measurement, 10 μl of the 2′-(4-Methylumbelliferyl)-α-D-N-acetylneuraminic acid (MUNANA) substrate (1 M NaAc, 10 mM CaCl2, 5% Butanol, and 2.5 mM MUNANA) was added. The plate was read in a BMG FLUOstar OPTIMA at 360 nm excitation and 460 nm emission wavelength, every 2 minutes for 1 h at 37° C.

Zanamivir competition assay. HEK293T cells were seeded at 3.5×10⁶ cells in 140 mm cell culture dishes and transfected with pCAXL-hPIV-3-HN, pCAXL-hPIV-1-HN, or empty pCAXL expression vector, in combination with peGFP-NLS, using polyethyleneimine (PEI) transfection reagent. One day after transfection, the cells were detached and resuspended in 1% BSA with or without Zanamivir (Merck) (at a concentration of 200 μg/ml in FIG. 9A, and concentrations of 500 μg/ml, 200 μg/ml, 100 μg/ml, 20 μg/ml, 2 μg/ml or 0 μg/mL in FIG. 9B). After 45 minutes of incubation, an equal volume of VHHs (0.1 μg/ml) or alpaca plasma or serum (1/50) was added and the cells were incubated for 10 to 15 minutes. One hour later, the cells were washed once with PBS/BSA, stained, and analyzed by flow cytometry using a BD® LSR II Flow Cytometer as described in the paragraph ‘Binding of VHHs to HEK293T cells expressing hPIV proteins.

Binding of VHHs to hPIV-1 HN and hPIV-3 HN muteins. Two mutations were introduced at positions 275 and 277 in hPIV-3 HN: D275N and R277T. Vice versa these amino acid residues at the corresponding positions in hPIV-1 HN were mutated into D and R, respectively (see FIG. 11A). The mutations were introduced into the pCAXL-hPIV-1-HN and pCAXL-hPIV-3-HN expression vectors by fusion PCR. HEK293T cells were then transfected with expression vectors for wild-type and mutant hPIV-1-HN (HN1 and mut HN1) and wild-type and mutant hPIV-3-HN (HN3 and mut HN3), or empty vector, and binding of monovalent and bivalent VHHs was assessed by flow cytometry, all as described in the paragraph “Binding of VHHs to HEK293T cells expressing hPIV proteins”. Concentrations used were 1 or 0.1 μg/mL for VHHs and 1/100 and 1/1000 dilution for serum.

Binding of VHHs to hPIV-2/-4a/-4b HN. Binding to hPIV-2/-4a/-4b HN was performed using flow cytometry as has been described above in ‘Binding of VHHs to HEK293T cells expressing hPIV proteins’. Binding of VHH-Fc was detected with anti-human IgG-Alexa633 (Invitrogen, A21091).

Mice. Specific pathogen-free, female DBA/2J mice were obtained from Janvier. The mice were housed in a temperature-controlled environment with 12h light/dark cycles; food and water were provided at libitum. The animal facility operates under the Flemish Government License Number LA1400536. All experiments were done under conditions specified by law and authorized by the Institutional Ethical Committee on Experimental Animals (Ethical application EC2023-027).

Administration of VHH-Fc and SeV/52 in mice. Seven weeks old DBA/2J mice, were randomly distributed in three experimental groups of five animals. A2R3-h60-Fc (100 μg), immune serum (50 μl) from SeV convalescent mice, or PBS was administered intraperitoneally in a total volume of 100 μl. One day later, the mice were slightly anesthetized by isoflurane before intranasal inoculation with 1000 PFU of SeV/52 in 50 μl. Mice were weighed daily during the experiment.

Determination of lung viral titers by plaque assay. Five days after challenge, the mice were sacrificed, lungs were removed and homogenized by vigorous shaking with a Mixer Mill 2000 (Retsch) in the presence of a sterile metal bead in 1 ml PBS supplemented with 1% penicillin and 1% streptomycin. Lung homogenates were cleared by centrifugation at 4° C. for 10 minutes at 1300 g and used to titrate the virus by plaque assay. In this assay a dilution series of the cleared lung homogenates was made in DMEM medium containing high glucose, GlutaMAX Supplement™, pyruvate (Gibco, 31966021) and non-essential amino acids (Invitrogen, Carlsbad, California). This dilution series was subsequently added to Vero (E6) TMPRSS2 cells in a 96-well plate. After three hours of incubation at 37° C., the inoculum was gently washed away and replaced with the above-mentioned DMEM supplemented with 0.6% avicel RC-581 (FMC Biopolymers). Three days after infection, immunostaining of the viral plaques was performed using goat anti-Parainfluenza type 1 (ab20791, Abcam) and peroxidase AffiniPure donkey anti-goat (705-035-003, Jackson Immunoresearch). The plaques were visualized by applying TrueBlue peroxidase substrate (KPL, Gaithersburg) and counted.

Determination of lung viral titer by RT-qPCR. To determine the lung SeV load by RT-qPCR, total RNA from the cleared lung homogenates was prepared by using the NucleoSpin RNA virus kit (Macherey-Nagel) according to the manufacturer's instructions. Next, cDNA was prepared by the use of random hexamer primers and the Transcriptor First Strand cDNA Synthesis kit (Roche). The relative levels of genomic SeV F cDNA were determined by RT-qPCR using primers specific for the SeV F gene (5′ AGA GAC ATA GCG CTC ATC AAA G 3′ and 5′ CTG GAG TGT CTT TAG AGC AAG AA 3′) using a LightCycler 480 II. The RT-qPCR data were normalized to HPRT1 and UBC mRNA levels, using the qbase+ software.

Production of recombinant hPIV-1 and -3 HN. HEK293s cells were transfected with plasmid encoding hPIV-1 or -3 HN N-terminally linked to either dimeric GCN4 (dGCN4) or tetrameric GCN4 (tetraGCN4). Transfections were performed with a 1:1 mixture of ExCell-293 (Sigma) and Freestyle-293 (Life Technologies) supplemented with 2 mM Glutamax. The proteins were purified from the cell supernatants using a StrepTrap XT column (Cytiva) followed by size-exclusion chromatography (Superdex 200 10/300 GL) in PBS.

Binding of VHH and VHH-Fc to recombinant hPIV-1 and -3 HN. The binding of A2R3-60-60 and A2R3-h60-Fc to hPIV-1/-3 HN was tested in ELISA. Microtiter plates (F96 Maxisorp, Nunc) were coated with 50 ng of each HN protein or BSA, or commercial recombinant hPIV-3 HN protein (Sino Biological 40801-V07B) in PBS. After washing, the plates were blocked with 4% milk in PBS, after which they were washed again. A 1/3 dilution series of A2R3-60-60 or A2R3-h60-Fc was then applied to the protein-coated wells. Bound A2R3-60-60 was detected with an anti-llama antibody (1/2000, A160-100A, ImTec diagnostics) and peroxidase AffiniPure donkey anti-goat (1/2000, 705-035-003, Jackson Immunoresearch). Bound h60-Fc was detected with anti-human IgG peroxidase linked (1/2000, A8792, Sigma). After washing, TMB substrate (Tetra metylbenzidine, BD OptEIA™) was added to every well. The reaction was stopped by addition of 100 aL of iM H₂SO₄ after which the absorbance was measured with an iMark Microplate Absorbance Reader (Bio Rad).

Protein stability measurement. An Uncle device (Unchained Labs) was used to measure full spectrum fluorescence and SLS at 266 nm and 473 nm at temperatures ranging from 25 to 95° C. of the samples at 1 mg/ml. Fluorescence and SLS data were analyzed using Uncle Analysis software to generate Tm, Tagg, and average particle size values.

Statistical analysis. Graphpad Prism 10 was used for statistical analysis, and the two-sided Mann-Whitney U-test to evaluate differences between two groups.

### SEQUENCE LISTING

SEQ ID NO: 1-4: monovalent VHH amino acid sequences ofIA23-60, A23-59, A23-57 and A23-55.
SEQ ID NO: 5-8: bivalent VHH amino acid sequences of A21R3-60, A21R3-59, A21R3-57 and A21R3-55, with a GS-linker.
SEQ ID NO:9-12: monovalent VHH nucleotide coding sequences encoding SEQ ID NO: 1-4, resp.
SEQ ID NO:13-16: bivalent VHH nucleotide coding sequences encoding SEQ ID NO: 5-8, resp.

TABLE 1
CDR sequences of hPIV-3 specific VHHs according to Chothia annotation.

| | FL SEQ | | SEQ ID | | SEQ ID | | SEQ ID |
|---|---|---|---|---|---|---|---|
| VHH | ID NO: | CDR1 | NO: | CDR2 | NO: | CDR3 | NO: |
| A2R3-60 | 1 | GTTVSIN | 17 | TTGGV | 21 | VASPSWGLYDDY | 25 |
| A2R3-59 | 2 | GSISGIN | 18 | TSGGS | 22 | SSTPSWGLYDDY | 26 |
| A2R3-57 | 3 | GSIFSTD | 19 | TSGGS | 23 | HQTTGWGLYNDY | 27 |
| A2R3-55 | 4 | GSIDRID | 20 | TTGGI | 24 | QGAPSWGLYEDY | 28 |

TABLE 2
FR sequences of hPIV-3 specific VHHs, wherein CDRs are annotated according to Chothia.

| | FL | | | | | | | | |
|---|---|---|---|---|---|---|---|---|---|
| | SEQ ID | | SEQ ID | | SEQ ID | | SEQ ID | | SEQ ID |
| VHH | NO: | FR1 | NO: | FR2 | NO: | FR3 | NO: | FR4 | NO: |
| A2R3- | 1 | QVQLQES | 29 | GMGWY | 33 | TNYADSVKGRF | 37 | WGQGT | 41 |
| 60 | | GGGLVQP | | RQAPGK | | TISMDNAKKTV | | QVTVSS | |
| | | GGSLRLD | | QREFVA | | ALQMNSLKPE | | | |
| | | CAAS | | LS | | DTAVYYCNA | | | |
| A2R3- | 2 | QVQLQES | 30 | GMGWY | 34 | TNYADSVKGRF | 38 | WGQGT | 41 |
| 59 | | GGGLVQP | | RQAPGK | | TISRDNAKNTV | | QVTVSS | |
| | | GGSLRLSC | | QRELVA | | DLQMNSLQPE | | | |
| | | AAS | | AI | | DTAVYYCYV | | | |
| A2R3- | 3 | QVQLQES | 31 | AMGWY | 35 | TNYVDSVKGRF | 39 | WGQGT | 41 |
| 57 | | GGGLVQP | | RQPPGK | | TISRDNAKNTV | | QVTVSS | |
| | | GGSLRLSC | | QRELVA | | YLQMNSLKPED | | | |
| | | AAS | | TI | | TAVYYCAL | | | |
| A2R3- | 4 | QVQLQES | 32 | AMGWS | 36 | TNYAVSVKGRF | 40 | WGQGT | 41 |
| 55 | | GGGLVQA | | RQAPGK | | TISRDNAKNTV | | QVTVSS | |
| | | GGSLRLSC | | QRELVA | | YLQMNSLKPED | | | |
| | | AAS | | TI | | TAVYYCNA | | | |

>SEQ ID NO: 42: FR1 consensus sequence
xVQLxESGGGLVQxGGSLRLxCAAS

wherein: x1: Q/D/E; x5: Q/V; x14: P/A; x21: D/S

>SEQ ID NO: 43: FR2 consensus sequence
xMGWxRQxPGKQRExVAxx

wherein: x1: G/A; x5: Y/S; x8: A/P; x15: F/L; x18: L/A/T; x19: S/I

>SEQ ID NO: 44: FR3 consensus sequence
TNYxxSVKGRFTISxDNAKxTVxLQMNSLxPEDTAVYYCxx

wherein: x4: A/V; x5: D/V; x15: M/R; x20: K/N; x23: A/D/Y; x30: K/Q/R; x40: N/Y/A; x41: A/V/L

SEQ ID NO: 45: FR4 consensus sequence
WGQGTxVTVSS

wherein x is Q or L
SEQ ID NO:46: Flexible spacer sequence
SEQ ID NO:47: MP075 primer
SEQ ID NO:48-50: HN fragments as shown in FIG. 11.
SEQ ID NO:51: hPIV-3 Hemagglutinin-neuraminidase amino acid sequence (as present in the expression vector)
SEQ ID NO: 52: humanized A2R3-60-60 bivalent construct version 1
SEQ ID NO: 53: humanized A2R3-59-59 bivalent construct version 1
SEQ ID NO: 54: humanized A2R3-57-57 bivalent construct version 1
SEQ ID NO: 55: humanized A2R3-60-Fc construct version 1
SEQ ID NO: 56: humanized A2R3-59-Fc construct version 1
SEQ ID NO: 57: humanized A2R3-57-Fc construct version 1
SEQ ID NO: 58: humanized A2R3-60-60 construct version 2(G33A)
SEQ ID NO: 59: humanized A2R3-59-59 construct version 2(G33A)
SEQ ID NO: 60: humanized A2R3-60-Fc construct version 2(G33A)
SEQ ID NO: 61: humanized A2R3-59-Fc construct version 2(G33A)
SEQ ID NO: 62-63: VHH fw/rev primers
SEQ ID NO: 64: humanized A2R3-60 monovalent VHH sequence version 1
SEQ ID NO: 65: humanized A2R3-59 monovalent VHH sequence version 1
SEQ ID NO: 66: humanized A2R3-57 monovalent VHH sequence version 1
SEQ ID NO: 67: humanized A2R3-60 monovalent VHH sequence version 1(1E)
SEQ ID NO: 68: humanized A2R3-59 monovalent VHH sequence version 1(1E)
SEQ ID NO: 69: humanized A2R3-57 monovalent VHH sequence version 1(1E)
SEQ ID NO:70: A2R3-60 (G33A) VHH amino acid sequence
SEQ ID NO:71: A2R3-59 (G33A) VHH amino acid sequence

### REFERENCES

1. Rafeek RAM, Divarathna MVM, Noordeen F. A review on disease burden and epidemiology of childhood parainfluenza virus infections in Asian countries. Rev Med Virol. 2021 March; 31(2):e2164
2. Zhao H, Harris R J, Ellis J, Donati M, Pebody R G. Epidemiology of parainfluenza infection in England and Wales, 1998-2013: any evidence of change?Epidemiol Infect. 2017 April; 145(6):1210-1220
3. Weinberg et al. New Vaccine Surveillance Network. Parainfluenza virus infection of young children: estimates of the population-based burden of hospitalization. J Pediatr. 2009 May; 154(5):694-9
4. Wang et al. Respiratory Virus Global Epidemiology Network. Global burden of acute lower respiratory infection associated with human parainfluenza virus in children younger than 5 years for 2018: a systematic review and meta-analysis. Lancet Glob Health. 2021 August; 9(8):e1077-e1087
5. Branche A R, Falsey A R. Parainfluenza Virus Infection. Semin Respir Crit Care Med. 2016 August; 37(4):538-54.
6. Glezen W P, Greenberg S B, Atmar R L, Piedra PA, Couch R B. Impact of respiratory virus infections on persons with chronic underlying conditions. JAMA. 2000 Jan. 26; 283(4):499-505
7. Nichols W G, Corey L, Gooley T, Davis C, Boeckh M. Parainfluenza virus infections after hematopoietic stem cell transplantation: risk factors, response to antiviral therapy, and effect on transplant outcome. Blood. 2001 Aug. 1; 98(3):573-8
8. Maykowski et al. Seasonality and clinical impact of human parainfluenza viruses. Influenza Other Respir Viruses. 2018 November; 12(6):706-716
9. Fry A M, Curns A T, Harbour K, Hutwagner L, Holman R C, Anderson L. Seasonal trends of human parainfluenza viral infections: United States, 1990-2004. Clin Infect Dis. 2006 Oct. 15; 43(8):1016-22
10. Marcink T C, Wang T, des Georges A, Porotto M, Moscona A. Human parainfluenza virus fusion complex glycoproteins imaged in action on authentic viral surfaces. PLoS Pathog. 2020 Sep. 21; 16(9):e1008883
11. Stewart-Jones et al. Structure-based design of a quadrivalent fusion glycoprotein vaccine for human parainfluenza virus types 1-4. Proc Natl Acad Sci USA. 2018 Nov. 27; 115(48):12265-12270
12. Boonyaratanakornkit et al. Protective antibodies against human parainfluenza virus type 3 infection. MAbs. 2021 January-December; 13(1):1912884
13. Matsunaga et al. Wheat germ cell-free system-based production of hemagglutinin-neuraminidase glycoprotein of human parainfluenza virus type 3 for generation and characterization of monoclonal antibody. Front Microbiol. 2014 May 13; 5:208
14. Goswami K K, Russell W C. Monoclonal antibodies against human paramyxovirus type 3 and against SV5 virus: preparation and preliminary characterization. J Gen Virol. 1983 August; 64 (Pt 8):1663-72
15. Orvell C, Rydbeck R, Love A. Immunological relationships between mumps virus and parainfluenza viruses studied with monoclonal antibodies. J Gen Virol. 1986 September; 67 (Pt 9):1929-39
16. Rydbeck R, Love A, Norrby E. Protective effects of monoclonal antibodies against parainfluenza virus type 3-induced brain infection in hamsters. J Gen Virol. 1988 May; 69 (Pt 5):1019-24
17. Lee M E, DeLoache W C, Cervantes B, Dueber J E. A Highly Characterized Yeast Toolkit for Modular, Multipart Assembly. ACS Synth Biol. 2015 Sep. 18; 4(9):975-86
18. Lin-Cereghino J, Wong W W, Xiong S, Giang W, Luong L T, Vu J, Johnson S D, Lin-Cereghino G P. Condensed protocol for competent cell preparation and transformation of the methylotrophic yeast Pichia pastoris. Biotechniques. 2005 January; 38(1):44, 46, 48
19. Piga N, Kessler N, Layani M P, Aymard M. Correlation between the reactivity patterns of monoclonal antibodies to distinct antigenic sites on HN glycoprotein and their protective abilities in Sendai (6/94) virus infection. Arch Virol. 1990; 110(3-4):179-93
20. Seo S, Xie H, Campbell A P, Kuypers J M, Leisenring W M, Englund J A, Boeckh M. Parainfluenza virus lower respiratory tract disease after hematopoietic cell transplant: viral detection in the lung predicts outcome. Clin Infect Dis. 2014 May; 58(10):1357-68
21. Rossey 1, Hsieh C L, Sedeyn K, Ballegeer M, Schepens B, Mclellan J S, Saelens X. A vulnerable, membrane-proximal site in human respiratory syncytial virus F revealed by a prefusion-specific single-domain antibody. J Virol. 2021 Mar. 10; 95(11):e02279-20
22. Tabatabai et al. Parainfluenza virus infections in patients with hematological malignancies or stem cell transplantation: Analysis of clinical characteristics, nosocomial transmission and viral shedding. PLoS One. 2022 Jul. 29; 17(7):e0271756.
23. Greengard, O., Poltoratskaia, N., Leikina, E. Zimmerberg, J. & Moscona, A. (2000). The antiinfluenza virus agent 4-GU-DANA (zanamivir) inhibits cell fusion mediated by human parainfluenza virus and influenza virus HA. J. Virol. 74, 11108-11114.
24. Ambrose M W, Wyde P R, Ewasyshyn M, Bonneau A M, Caplan B, Meyer H L, Klein M. (1991). Evaluation of the immunogenicity and protective efficacy of a candidate parainfluenza virus type 3 subunit vaccine in cotton rats. Vaccine. 9(7):505-11.
25. Ray R, Glaze B J, Compans R W. (1988). Role of individual glycoproteins of human parainfluenza virus type 3 in the induction of a protective immune response. J Virol. 62(3):783-7.
26. Spriggs M K, Murphy B R, Prince G A, Olmsted R A, Collins P L. (1987). Expression of the F and HN glycoproteins of human parainfluenza virus type 3 by recombinant vaccinia viruses: contributions of the individual proteins to host immunity. J Virol. 61(11):3416-23.

1. An antigen-binding protein comprising a means for specifically binding human parainfluenza virus (hPIV) serotype 3 haemagglutinin-neuraminidase (HN), wherein the antigen-binding protein is neutralizing for hPIV-3 and Sendai virus.

2. The antigen-binding protein of claim 1, wherein the antigen-binding protein competes for binding to hPIV-3 HN with zanamivir.

3. The antigen-binding protein of claim 1, which neutralizes hPIV-3 and Sendai virus with an IC₅₀ value of 1 μM or lower, preferably with an IC₅₀ value below 30 nM.

4. The antigen-binding protein of claim 1, which further also neutralizes hPIV-1 virus.

5. The antigen-binding protein of claim 1, which neutralizes hPIV-3, Sendai virus and hPIV-1 with an IC₅₀ value of 1.5 M or lower, preferably with an IC₅₀ value below 30 nM.

6. The antigen-binding protein of claim 1, comprising an immunoglobulin single variable domain (ISVD) specifically binding hPIV-3 HN protein.

7. The antigen-binding protein of claim 1, comprising:
an ISVD comprising the complementarity determining regions (CDRs) as presented in any of SEQ ID NOs: 1-4, 70, or 71, wherein the CDRs are annotated according to Kabat, MacCallum, IMGT, AbM, or Chothia, or comprising an ISVD comprising a sequence wherein:
CDR1 comprises SEQ ID NO: 17, CDR2 comprises SEQ ID NO: 21, and CDR3 comprises SEQ ID NO: 25;
CDR1 comprises SEQ ID NO: 18, CDR2 comprises SEQ ID NO: 22, and CDR3 comprises SEQ ID NO: 26;
CDR1 comprises SEQ ID NO: 19, CDR2 comprises SEQ ID NO: 23, and CDR3 comprises SEQ ID NO: 27; or
CDR1 comprises SEQ ID NO: 20, CDR2 comprises SEQ ID NO: 24, and CDR3 comprises SEQ ID NO: 28.

8. The antigen-binding protein of claim 7, comprising an ISVD comprising the Framework regions (FRs) wherein FR1 comprises SEQ ID NO:42, FR2 comprises SEQ ID NO: 43, FR3 comprises SEQ ID NO: 44, FR4 comprises SEQ ID NO: 45, or an ISVD comprising the Framework regions (FRs) as presented in any of SEQ ID NOs: 1-4, 70 or 71.

9. The antigen-binding protein of claim 1 comprising an immunoglobulin single variable domain (ISVD),
wherein the ISVD comprises;
a sequence selected from the group of sequences of SEQ ID NOs: 1-4, or
a functional variant of any one thereof with at least 90% amino acid identity over the full length of the ISVD sequence wherein the non-identical amino acids are located in one or more Framework residues, or
a humanized variant of any one thereof, or
a humanized variant wherein:
a. at least one of the following substitutions is made in SEQ ID NO:1: Q1D/E, Q5V, D21S, G33A, M71R, K76N, A79Y, K83R, Q108L;
b. at least one of the following substitutions is made in SEQ ID NO:2: Q1D/E, Q5V, G33A, D79Y, Q83R, Q108L;
c. at least one of the following substitutions is made in SEQ ID NO:3: Q1D/E, Q5V, P40A, V60A, K83R, Q108L; or
a humanized variant comprising any one of SEQ ID NOs: 64-71.

10. The antigen-binding protein of claim 1, which is a multivalent or multispecific antigen-binding agent, preferably a bivalent or bispecific antigen-binding agent, which may be in an Fc fusion or an antibody format.

11. The antigen-binding protein of claim 10, which comprises one or more sequences selected from the group of sequences of SEQ ID NOs: 5-8, or a functional variant of any one thereof with at least 90% amino acid identity over the full length of the ISVD sequence wherein the non-identical amino acids are located in one or more Framework residues, or a humanized variant of any one thereof, or a multivalent or multispecific antigen-binding agent which comprises a humanized variant of any one of the SEQ ID NOs: 1-4, 70, 71, or comprising the humanized variants of any one of SEQ ID NOs:64-71, such as the antigen-binding protein comprising any one of SEQ ID NOs: 52-61.

12. The antigen-binding protein of claim 1, which is labelled, or conjugated to a functional moiety, such as a therapeutic moiety or a half-life extension.

13. A nucleic acid molecule encoding the antigen-binding protein of claim 1.

14. The antigen-binding protein of claim 1, wherein the antigen binding protein is comprised in a pharmaceutical composition having one or more of a further therapeutically active agent, a pharmaceutically acceptable carrier, adjuvant, excipient or diluent.

15. A method for the treatment of human parainfluenza infection, acute respiratory tract infections, croup, pneumonia, and exacerbations of chronic obstructive pulmonary disease in a subject, the method comprising: administering to the subject the antigen-binding protein of claim 1.


SEQUENCE LISTING (71 sequences)

SEQ ID NO: 1 - protein, 120 aa, synthetic construct
QVQLQESGGGLVQPGGSLRLDCAASGTTVSINGMGWYRQAPGKQREFVALSTTGGVTNYADSVKGRFTISMDNAKKTVALQMNSLKPEDTAVYYCNAVASPSWGLYDDYWGQGTQVTVSS

SEQ ID NO: 2 - protein, 120 aa, synthetic construct
QVQLQESGGGLVQPGGSLRLSCAASGSISGINGMGWYRQAPGKQRELVAAITSGGSTNYADSVKGRFTISRDNAKNTVDLQMNSLQPEDTAVYYCYVSSTPSWGLYDDYWGQGTQVTVSS

SEQ ID NO: 3 - protein, 120 aa, synthetic construct
QVQLQESGGGLVQPGGSLRLSCAASGSIFSTDAMGWYRQPPGKQRELVATITSGGSTNYVDSVKGRFTISRDNAKNTVYLQMNSLKPEDTAVYYCALHQTTGWGLYNDYWGQGTQVTVSS

SEQ ID NO: 4 - protein, 120 aa, synthetic construct
QVQLQESGGGLVQAGGSLRLSCAASGSIDRIDAMGWSRQAPGKQRELVATITTGGITNYAVSVKGRFTISRDNAKNTVYLQMNSLKPEDTAVYYCNAQGAPSWGLYEDYWGQGTQVTVSS

SEQ ID NO: 5 - protein, 255 aa, synthetic construct
QVQLQESGGGLVQPGGSLRLDCAASGTTVSINGMGWYRQAPGKQREFVALSTTGGVTNYADSVKGRFTISMDNAKKTVALQMNSLKPEDTAVYYCNAVASPSWGLYDDYWGQGTQVTVSSGGGGSGGGGSGGGGSQVQLQESGGGLVQPGGSLRLDCAASGTTVSINGMGWYRQAPGKQREFVALSTTGGVTNYADSVKGRFTISMDNAKKTVALQMNSLKPEDTAVYYCNAVASPSWGLYDDYWGQGTQVTVSS

SEQ ID NO: 6 - protein, 255 aa, synthetic construct
QVQLQESGGGLVQPGGSLRLSCAASGSISGINGMGWYRQAPGKQRELVAAITSGGSTNYADSVKGRFTISRDNAKNTVDLQMNSLQPEDTAVYYCYVSSTPSWGLYDDYWGQGTQVTVSSGGGGSGGGGSGGGGSQVQLQESGGGLVQPGGSLRLSCAASGSISGINGMGWYRQAPGKQRELVAAITSGGSTNYADSVKGRFTISRDNAKNTVDLQMNSLQPEDTAVYYCYVSSTPSWGLYDDYWGQGTQVTVSS

SEQ ID NO: 7 - protein, 255 aa, synthetic construct
QVQLQESGGGLVQPGGSLRLSCAASGSIFSTDAMGWYRQPPGKQRELVATITSGGSTNYVDSVKGRFTISRDNAKNTVYLQMNSLKPEDTAVYYCALHQTTGWGLYNDYWGQGTQVTVSSGGGGSGGGGSGGGGSQVQLQESGGGLVQPGGSLRLSCAASGSIFSTDAMGWYRQPPGKQRELVATITSGGSTNYVDSVKGRFTISRDNAKNTVYLQMNSLKPEDTAVYYCALHQTTGWGLYNDYWGQGTQVTVSS

SEQ ID NO: 8 - protein, 255 aa, synthetic construct
QVQLQESGGGLVQAGGSLRLSCAASGSIDRIDAMGWSRQAPGKQRELVATITTGGITNYAVSVKGRFTISRDNAKNTVYLQMNSLKPEDTAVYYCNAQGAPSWGLYEDYWGQGTQVTVSSGGGGSGGGGSGGGGSQVQLQESGGGLVQAGGSLRLSCAASGSIDRIDAMGWSRQAPGKQRELVATITTGGITNYAVSVKGRFTISRDNAKNTVYLQMNSLKPEDTAVYYCNAQGAPSWGLYEDYWGQGTQVTVSS

SEQ ID NO: 9 - DNA, 360 bp, synthetic construct
  source 1..360: mol_type = other DNA
caggtgcagctgcaggagtctggaggaggcttggtgcagcctgggggctctctgagactcgactgtgcagcctctggaaccacagtcagtatcaatggcatgggctggtaccgccaggctccagggaagcagcgcgagttcgtcgcacttagtactactggtggtgtcacaaactatgcagactccgtgaagggccgattcaccatctccatggacaacgccaagaagacggtggctctacaaatgaacagcttgaaacctgaggacacggccgtctattactgtaatgcagtggcctcaccgagttggggcctatatgatgactactggggccaggggacccaggtcaccgtgtcctca

SEQ ID NO: 10 - DNA, 360 bp, synthetic construct
  source 1..360: mol_type = other DNA
caggtgcagctgcaggagtctgggggaggcttggtgcagcctgggggctctctgagactctcctgtgcagcctctggaagcatcagcggtatcaatggcatgggctggtaccgccaggctccagggaagcagcgcgagttggtcgccgctattactagtggtggtagcacaaactatgcagactccgtgaagggccgattcaccatctccagagacaacgccaagaacacggtagatctgcaaatgaacagcctgcaacctgaggatacggccgtctattactgttatgtctcctccacgccttcgtggggactgtatgatgactactggggccaggggacccaggtcaccgtgtcctca

SEQ ID NO: 11 - DNA, 360 bp, synthetic construct
  source 1..360: mol_type = other DNA
caggtgcagctgcaggagtctgggggaggtttggtgcagcctgggggctctctgagactctcctgtgcagcctctggaagcatcttcagtaccgatgccatgggctggtatcgccagcctccagggaagcagcgcgaattggtcgcaactattactagtggtggtagcacaaactatgtagactccgtgaagggccgattcaccatctccagagacaacgccaagaacacggtgtatctgcaaatgaacagcctgaaacctgaggacacggccgtctattactgtgcgctacatcagacgacgggttggggcctctataatgactactggggccaggggacccaggtcaccgtgtcctca

SEQ ID NO: 12 - DNA, 360 bp, synthetic construct
  source 1..360: mol_type = other DNA
caggtgcagctgcaggagtctgggggaggcttggtgcaggctgggggctctctgagactctcctgtgcagcctctggaagcatcgaccgtatcgatgccatgggctggtcccgccaggctccagggaagcagcgcgaattggtcgctactattactacaggtggtatcacaaactatgcagtctccgtgaagggccgattcaccatctccagagacaacgccaagaacacggtgtatctgcaaatgaacagcctgaaacctgaggacacggccgtctattactgtaatgctcagggggcgccaagttggggcctctacgaggactattggggccaggggacccaggtcaccgtgtcctca

SEQ ID NO: 13 - DNA, 765 bp, synthetic construct
  source 1..765: mol_type = other DNA
caggttcaattgcaagaatccggtggtggtttggttcaaccaggtggttctttgagattggactgtgctgcttccggtactaccgtttccattaacggtatgggttggtacagacaggctccaggtaagcaaagagagttcgttgctttgtccactaccggtggtgttaccaactacgctgattctgtcaagggtagattcaccatctccatggacaacgccaagaaaactgttgccttgcagatgaactccctgaagccagaagataccgccgtttactactgtaacgctgttgcttctccatcctggggtctgtacgatgattattggggtcaaggtactcaggtcactgtttcctctggtggtggtggatctggtggcggaggtagcggtggtggcggttctcaagttcaactacaagaatctggcggaggacttgttcagcctggtggtagtttgagacttgattgtgcagcttctggaaccaccgtgtctatcaatggaatgggatggtatagacaagcccctggaaagcagagagaatttgtcgccttgtctacaactggtggcgtcactaattacgccgactctgttaagggtcgtttcactattagcatggataacgctaaaaagaccgtcgctctgcaaatgaattctttgaagcctgaggacactgctgtctactactgcaatgcagttgcaagtccttcttggggcttgtacgacgactactggggacagggaactcaagttactgtttcttct

SEQ ID NO: 14 - DNA, 765 bp, synthetic construct
  source 1..765: mol_type = other DNA
caagttcaattgcaagaatctggtggtggtttggttcaaccaggtggttctttgagattgtcttgtgctgcttctggttctatttctggtattaacggtatgggttggtatagacaagctccaggtaaacaaagagaattggttgctgctattacttctggtggttctactaattatgccgattctgttaagggtcgtttcaccatttctagagataacgctaagaacaccgttgacttgcagatgaattctctacaaccagaagataccgctgtttactactgttacgtttcttctactccatcttggggcttgtatgatgattattggggtcaaggtactcaggttactgtttcatctggtggcggtggttcaggtggtggtggatctggcggtggcggttcacaagtacaactacaagaaagcggtggtggtctagtacagcctggtggtagtttaagattatcatgtgcagcatccggttccatctctggtatcaatggaatgggatggtacaggcaagcacctggtaagcaacgtgaattagttgcagcaattacaagtggtggttcaacaaactacgctgattcagtaaaaggtagattcaccatctcaagagacaatgccaagaatactgtagacctgcaaatgaactcattgcagcctgaagatactgcagtctattattgctatgttagctctacaccatcatggggtctatacgatgactactggggtcagggaacccaagttacagtttctagt

SEQ ID NO: 15 - DNA, 765 bp, synthetic construct
  source 1..765: mol_type = other DNA
caggttcaattgcaagaatccggtggtggtttggttcaaccaggtggttctttgagattgtcctgtgctgcttccggttccattttctctactgatgctatgggttggtacagacagccaccaggtaagcaaagagagttggttgctactatcacttccggtggttccaccaactacgttgactctgttaagggtagattcaccatctccagagacaacgccaagaacaccgtttacttgcagatgaactccctgaagccagaggacactgctgtttactactgtgccttgcaccaaactactggttggggtctgtacaacgattactggggtcaaggtactcaggttactgtttcttctggtggtggtggatctggtggcggaggtagcggtggtggcggttctcaagttcaactacaagaatctggcggaggacttgttcagcctggtggtagtttgagactttcttgtgcagcctctggatccatcttttccacagatgcaatgggctggtataggcaacctcctggtaaacagagagaactggttgccactattacctctggtggcagcactaactacgtcgattctgtcaaaggacgtttcactatctctcgtgataacgctaagaatactgtctaccttcaaatgaacagcttgaagcctgaggataccgccgtctattattgtgctctgcatcagactacaggatggggcttgtacaatgactattggggacagggaacccaagttaccgtttcctct

SEQ ID NO: 16 - DNA, 765 bp, synthetic construct
  source 1..765: mol_type = other DNA
caggttcaattgcaagaatccggtggtggtttggttcaagctggtggttctttgagattgtcctgtgctgcttctggttccatcgacagaattgatgctatgggttggtccagacaagctccaggtaagcaaagagagttggttgctactatcactaccggtggtatcaccaactacgccgtttctgttaagggtagattcaccatctccagagacaacgccaagaacaccgtttacttgcagatgaactccctgaagccagaggacactgctgtttactactgtaacgctcaaggtgctccatcttggggtctgtacgaagattattggggtcagggtactcaggttactgtttcttctggtggtggtggatctggtggcggaggtagcggtggtggcggttctcaagttcaactacaagaatctggcggaggacttgtacaggctggtggatcacttagactttcttgtgctgcctccggatccattgatagaatcgacgcaatgggatggtctagacaggcacctggaaaacagagagaattggtcgctaccattaccactggcggaattacaaactacgctgtctctgtcaaaggccgtttcactatttctagggataacgctaagaatactgtctaccttcaaatgaacagcttgaagcctgaggataccgccgtctactattgcaatgcacagggtgcaccttcatggggactttacgaggactactggggacaaggtacacaagtcaccgtttcttct

SEQ ID NO: 17 - protein, 7 aa, synthetic construct
GTTVSIN

SEQ ID NO: 18 - protein, 7 aa, synthetic construct
GSISGIN

SEQ ID NO: 19 - protein, 7 aa, synthetic construct
GSIFSTD

SEQ ID NO: 20 - protein, 7 aa, synthetic construct
GSIDRID

SEQ ID NO: 21 - protein, 5 aa, synthetic construct
TTGGV

SEQ ID NO: 22 - protein, 5 aa, synthetic construct
TSGGS

SEQ ID NO: 23 - protein, 5 aa, synthetic construct
TSGGS

SEQ ID NO: 24 - protein, 5 aa, synthetic construct
TTGGI

SEQ ID NO: 25 - protein, 12 aa, synthetic construct
VASPSWGLYDDY

SEQ ID NO: 26 - protein, 12 aa, synthetic construct
SSTPSWGLYDDY

SEQ ID NO: 27 - protein, 12 aa, synthetic construct
HQTTGWGLYNDY

SEQ ID NO: 28 - protein, 12 aa, synthetic construct
QGAPSWGLYEDY

SEQ ID NO: 29 - protein, 25 aa, synthetic construct
QVQLQESGGGLVQPGGSLRLDCAAS

SEQ ID NO: 30 - protein, 25 aa, synthetic construct
QVQLQESGGGLVQPGGSLRLSCAAS

SEQ ID NO: 31 - protein, 25 aa, synthetic construct
QVQLQESGGGLVQPGGSLRLSCAAS

SEQ ID NO: 32 - protein, 25 aa, synthetic construct
QVQLQESGGGLVQAGGSLRLSCAAS

SEQ ID NO: 33 - protein, 19 aa, synthetic construct
GMGWYRQAPGKQREFVALS

SEQ ID NO: 34 - protein, 19 aa, synthetic construct
GMGWYRQAPGKQRELVAAI

SEQ ID NO: 35 - protein, 19 aa, synthetic construct
AMGWYRQPPGKQRELVATI

SEQ ID NO: 36 - protein, 19 aa, synthetic construct
AMGWSRQAPGKQRELVATI

SEQ ID NO: 37 - protein, 41 aa, synthetic construct
TNYADSVKGRFTISMDNAKKTVALQMNSLKPEDTAVYYCNA

SEQ ID NO: 38 - protein, 41 aa, synthetic construct
TNYADSVKGRFTISRDNAKNTVDLQMNSLQPEDTAVYYCYV

SEQ ID NO: 39 - protein, 41 aa, synthetic construct
TNYVDSVKGRFTISRDNAKNTVYLQMNSLKPEDTAVYYCAL

SEQ ID NO: 40 - protein, 41 aa, synthetic construct
TNYAVSVKGRFTISRDNAKNTVYLQMNSLKPEDTAVYYCNA

SEQ ID NO: 41 - protein, 11 aa, synthetic construct
WGQGTQVTVSS

SEQ ID NO: 42 - protein, 25 aa, synthetic construct
  VARIANT 1: note = Q, D or E
  VARIANT 5: note = Q or V
  VARIANT 14: note = P or A
  VARIANT 21: note = D or S
XVQLXESGGGLVQXGGSLRLXCAAS

SEQ ID NO: 43 - protein, 19 aa, synthetic construct
  VARIANT 1: note = G or A
  VARIANT 5: note = Y or S
  VARIANT 8: note = A or P
  VARIANT 15: note = F or L
  VARIANT 18: note = L, A or T
  VARIANT 19: note = S or I
XMGWXRQXPGKQREXVAXX

SEQ ID NO: 44 - protein, 41 aa, synthetic construct
  VARIANT 4: note = A or V
  VARIANT 5: note = D or V
  VARIANT 15: note = M or R
  VARIANT 20: note = K or N
  VARIANT 23: note = A, D or Y
  VARIANT 30: note = K, Q or R
  VARIANT 40: note = N, Y or A
  VARIANT 41: note = A, V or L
TNYXXSVKGRFTISXDNAKXTVXLQMNSLXPEDTAVYYCXX

SEQ ID NO: 45 - protein, 11 aa, synthetic construct
  VARIANT 6: note = Q or L
WGQGTXVTVSS

SEQ ID NO: 46 - protein, 15 aa, synthetic construct
GGGGSGGGGSGGGGS

SEQ ID NO: 47 - DNA, 20 bp, synthetic construct
  source 1..20: mol_type = other DNA
ttatgcttccggctcgtatg

SEQ ID NO: 48 - protein, 10 aa, Human parainfluenza virus 3
TPKVDERSDY

SEQ ID NO: 49 - protein, 10 aa, Sendai Virus
MPTVDERTDY

SEQ ID NO: 50 - protein, 10 aa, Human parainfluenza virus serotype 1
LPTVNETTDY

SEQ ID NO: 51 - protein, 572 aa, Human parainfluenza virus 3
MEYWKHTNHGKDAGNELETSTATHGNKLTNKITYILWTITLVLLSIVFIIVLINSIKSEKARESLLQDINNELMEVTEKIQVASDNTNDLIQSGVNTRLLTIQSHVQNYIPISLTQQISDLRKFISEITIRNDNQEVPPQRITHDVGIKPLNPDDFWRCTSGLPSLMKTPKIRSMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENAICNTTGCPGKTQRDCNQASHSPWFSDRRMVNSIIVVDKGLNSVPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNKTLSAGYTTTSCITHYNKGYCFHIVEINHKSLNTFQPMLFKTEIPKSCS

SEQ ID NO: 52 - protein, 255 aa, synthetic construct
DVQLVESGGGLVQPGGSLRLSCAASGTTVSINGMGWYRQAPGKQREFVALSTTGGVTNYADSVKGRFTISRDNAKNTVYLQMNSLRPEDTAVYYCNAVASPSWGLYDDYWGQGTLVTVSSGGGGSGGGGSGGGGSEVQLVESGGGLVQPGGSLRLSCAASGTTVSINGMGWYRQAPGKQREFVALSTTGGVTNYADSVKGRFTISRDNAKNTVYLQMNSLRPEDTAVYYCNAVASPSWGLYDDYWGQGTLVTVSS

SEQ ID NO: 53 - protein, 255 aa, synthetic construct
DVQLVESGGGLVQPGGSLRLSCAASGSISGINGMGWYRQAPGKQRELVAAITSGGSTNYADSVKGRFTISRDNAKNTVYLQMNSLRPEDTAVYYCYVSSTPSWGLYDDYWGQGTLVTVSSGGGGSGGGGSGGGGSEVQLVESGGGLVQPGGSLRLSCAASGSISGINGMGWYRQAPGKQRELVAAITSGGSTNYADSVKGRFTISRDNAKNTVYLQMNSLRPEDTAVYYCYVSSTPSWGLYDDYWGQGTLVTVSS

SEQ ID NO: 54 - protein, 255 aa, synthetic construct
DVQLVESGGGLVQPGGSLRLSCAASGSIFSTDAMGWYRQAPGKQRELVATITSGGSTNYADSVKGRFTISRDNAKNTVYLQMNSLRPEDTAVYYCALHQTTGWGLYNDYWGQGTLVTVSSGGGGSGGGGSGGGGSEVQLVESGGGLVQPGGSLRLSCAASGSIFSTDAMGWYRQAPGKQRELVATITSGGSTNYADSVKGRFTISRDNAKNTVYLQMNSLRPEDTAVYYCALHQTTGWGLYNDYWGQGTLVTVSS

SEQ ID NO: 55 - protein, 356 aa, synthetic construct
DVQLVESGGGLVQPGGSLRLSCAASGTTVSINGMGWYRQAPGKQREFVALSTTGGVTNYADSVKGRFTISRDNAKNTVYLQMNSLRPEDTAVYYCNAVASPSWGLYDDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 356 aa, synthetic construct
DVQLVESGGGLVQPGGSLRLSCAASGSISGINGMGWYRQAPGKQRELVAAITSGGSTNYADSVKGRFTISRDNAKNTVYLQMNSLRPEDTAVYYCYVSSTPSWGLYDDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 356 aa, synthetic construct
DVQLVESGGGLVQPGGSLRLSCAASGSIFSTDAMGWYRQAPGKQRELVATITSGGSTNYADSVKGRFTISRDNAKNTVYLQMNSLRPEDTAVYYCALHQTTGWGLYNDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 255 aa, synthetic construct
DVQLVESGGGLVQPGGSLRLSCAASGTTVSINAMGWYRQAPGKQREFVALSTTGGVTNYADSVKGRFTISRDNAKNTVYLQMNSLRPEDTAVYYCNAVASPSWGLYDDYWGQGTLVTVSSGGGGSGGGGSGGGGSDVQLVESGGGLVQPGGSLRLSCAASGTTVSINAMGWYRQAPGKQREFVALSTTGGVTNYADSVKGRFTISRDNAKNTVYLQMNSLRPEDTAVYYCNAVASPSWGLYDDYWGQGTLVTVSS

SEQ ID NO: 59 - protein, 255 aa, synthetic construct
DVQLVESGGGLVQPGGSLRLSCAASGSISGINAMGWYRQAPGKQRELVAAITSGGSTNYADSVKGRFTISRDNAKNTVYLQMNSLRPEDTAVYYCYVSSTPSWGLYDDYWGQGTLVTVSSGGGGSGGGGSGGGGSEVQLVESGGGLVQPGGSLRLSCAASGSISGINAMGWYRQAPGKQRELVAAITSGGSTNYADSVKGRFTISRDNAKNTVYLQMNSLRPEDTAVYYCYVSSTPSWGLYDDYWGQGTLVTVSS

SEQ ID NO: 60 - protein, 356 aa, synthetic construct
DVQLVESGGGLVQPGGSLRLSCAASGTTVSINAMGWYRQAPGKQREFVALSTTGGVTNYADSVKGRFTISRDNAKNTVYLQMNSLRPEDTAVYYCNAVASPSWGLYDDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 356 aa, synthetic construct
DVQLVESGGGLVQPGGSLRLSCAASGSISGINAMGWYRQAPGKQRELVAAITSGGSTNYADSVKGRFTISRDNAKNTVYLQMNSLRPEDTAVYYCYVSSTPSWGLYDDYWGQGTLVTVSSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - DNA, 38 bp, synthetic construct
  source 1..38: mol_type = other DNA
cttggctcttctgtgcagctgcaggagtctggrggagg

SEQ ID NO: 63 - DNA, 35 bp, synthetic construct
  source 1..35: mol_type = other DNA
tgatgctcttccgctgaggagacggtgacctgggt

SEQ ID NO: 64 - protein, 120 aa, synthetic construct
DVQLVESGGGLVQPGGSLRLSCAASGTTVSINGMGWYRQAPGKQREFVALSTTGGVTNYADSVKGRFTISRDNAKNTVYLQMNSLRPEDTAVYYCNAVASPSWGLYDDYWGQGTLVTVSS

SEQ ID NO: 65 - protein, 120 aa, synthetic construct
DVQLVESGGGLVQPGGSLRLSCAASGSISGINGMGWYRQAPGKQRELVAAITSGGSTNYADSVKGRFTISRDNAKNTVYLQMNSLRPEDTAVYYCYVSSTPSWGLYDDYWGQGTLVTVSS

SEQ ID NO: 66 - protein, 120 aa, synthetic construct
DVQLVESGGGLVQPGGSLRLSCAASGSIFSTDAMGWYRQAPGKQRELVATITSGGSTNYADSVKGRFTISRDNAKNTVYLQMNSLRPEDTAVYYCALHQTTGWGLYNDYWGQGTLVTVSS

SEQ ID NO: 67 - protein, 120 aa, synthetic construct
EVQLVESGGGLVQPGGSLRLSCAASGTTVSINGMGWYRQAPGKQREFVALSTTGGVTNYADSVKGRFTISRDNAKNTVYLQMNSLRPEDTAVYYCNAVASPSWGLYDDYWGQGTLVTVSS

SEQ ID NO: 68 - protein, 120 aa, synthetic construct
EVQLVESGGGLVQPGGSLRLSCAASGSISGINGMGWYRQAPGKQRELVAAITSGGSTNYADSVKGRFTISRDNAKNTVYLQMNSLRPEDTAVYYCYVSSTPSWGLYDDYWGQGTLVTVSS

SEQ ID NO: 69 - protein, 120 aa, synthetic construct
EVQLVESGGGLVQPGGSLRLSCAASGSIFSTDAMGWYRQAPGKQRELVATITSGGSTNYADSVKGRFTISRDNAKNTVYLQMNSLRPEDTAVYYCALHQTTGWGLYNDYWGQGTLVTVSS

SEQ ID NO: 70 - protein, 120 aa, synthetic construct
QVQLQESGGGLVQPGGSLRLDCAASGTTVSINAMGWYRQAPGKQREFVALSTTGGVTNYADSVKGRFTISMDNAKKTVALQMNSLKPEDTAVYYCNAVASPSWGLYDDYWGQGTQVTVSS

SEQ ID NO: 71 - protein, 120 aa, synthetic construct
QVQLQESGGGLVQPGGSLRLSCAASGSISGINAMGWYRQAPGKQRELVAAITSGGSTNYADSVKGRFTISRDNAKNTVDLQMNSLQPEDTAVYYCYVSSTPSWGLYDDYWGQGTQVTVSS